(19) United States
(12) Patent Application Publication
(10) Pub. No.: US 2017/0328917 A1
(43) Pub. Date: Nov. 16, 2017

(54) AUTISM-ASSOCIATED BIOMARKERS AND USES THEREOF

(71) Applicant: The Trustees of Columbia University in the City of New York, New York, NY (US)

(72) Inventors: Mady HORNIG, New York, NY (US); W. Ian LIPKIN, New York, NY (US)

(21) Appl. No.: 15/391,515

(22) Filed: Dec. 27, 2016

Related U.S. Application Data
(63) Continuation of application No. 14/474,523, filed on Sep. 2, 2014, now abandoned.
Application No. 14/474,523 is a continuation-in-part of application No. PCT/US2013/028589, filed on Mar. 1, 2013.
(60) Provisional application No. 61/605,567, filed on Mar. 1, 2012.

(51) Int. Cl. G01N 33/68; A61K 38/17; A61K 38/19; A61K 45/06; C12Q 1/68; A61K 31/593; A61N 5/06; C07K 16/18; A61K 39/395

(52) U.S. Cl. CPC G01N 33/6896; C07K 16/18; A61K 38/1722; A61K 38/19; A61K 39/3955; C12Q 1/6883; A61K 31/593; A61N 5/062; A61K 45/06; C12Q 2600/106; C12Q 2600/112; C12Q 2600/158; G01N 2800/2814

(57) ABSTRACT

The invention discloses biomarkers for human autism. The invention provides methods for treating, preventing, and diagnosing human autism and autism-related disorders.

This application is a continuation of U.S. Non-Provisional patent application Ser. No. 14/474,523, filed Sep. 2, 2014, which is a Continuation-In-Part of International Patent Application No. PCT/US2013/028589 filed Mar. 1, 2013, which claims priority of U.S. Provisional Patent Application No. 61/605,567, filed Mar. 1, 2012, the contents of which are hereby incorporated by reference in their entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under Grant No. U01 NS047537 awarded by the National Institutes of Health. The Government has certain rights in the invention.

### SEQUENCE LISTING

The instant application contains a Sequence Listing which has been submitted electronically in ASCII format and is hereby incorporated by reference in its entirety.

### BACKGROUND OF THE INVENTION

Autistic disorder is one of five pervasive developmental disorders defined in the Diagnostic and Statistical Manual of Mental Disorders, Fourth Edition, Text Revision DSM-IV-TR (2000). Autistic disorder is a developmental disorder of the human brain that manifests during infancy or childhood and is characterized by behavioral and social abnormalities that appear to be developmentally based (for example, impairments in social interaction and communication). In addition, autism interferes with imagination and the ability to reason. Autism is frequently associated with other disorders such as attention deficit/hyperactivity disorder (AD/HD) and can be associated with psychiatric symptoms such as anxiety and depression. In the last decade, autism diagnoses have increased by 300% to 500% in the United States and many other countries. A means of prevention and treatment is needed for this health crisis that addresses the underlying mechanisms leading to the development of autism versus those that merely address the symptoms.

Autism Spectrum Disorders (ASD) are a common group of neurodevelopmental disorders associated with atypical socialization, communication, and speech patterns. While the disease seems to have a genetic basis, no causal mutations have been identified. Pervasive developmental disorders (PDDs) are also part of the Autism Spectrum Disorders (ASDs). PDD is used to categorize children who do not meet the strict criteria for Autistic Disorder but who come close, either by manifesting atypical autism or by nearly meeting the diagnostic criteria in two or three of the key areas. Some of these children meet criteria for the ASD known as Asperger's Disorder (ASP), wherein language capacities are relatively spared compared to children with Autistic Disorder. Others meet criteria for the PDDs known as Childhood Disintegrative Disorder, which begins at a slightly later age than the other ASDs, or Rett's Disorder, which is related to a mutation in a DNA methylation binding protein gene called MeCP2 and usually occurs in girls. Diagnosis of ASDs is achieved solely by behavioral assays, and thus can usually only be done after ˜3 yrs of age. Since early detection has been associated with dramatically improved patient outcomes, the identification of biomarkers and other early detection tools is a major focus of research.

### SUMMARY OF THE INVENTION

The invention is based, at least in part, on the finding that increased levels in the Gc globulin GcF1 (which is a vitamin D binding protein) can serve as a biomarker for human Autism Spectrum Disorders. Specifically, this biomarker for ASD can be detected in a pregnant female, in a child of a human subject at birth or in the early years of life, or in an unborn human child.

Accordingly, in one aspect, the invention provides a method for detecting the presence of or a predisposition to autism or an autism spectrum disorder (ASD) in a human subject or a child of a human subject. In some embodiments, the method comprises: (a) obtaining a biological sample from a human subject; and (b) detecting whether or not there is an alteration in the expression of a Gc globulin protein in the subject as compared to a non-autistic subject. In other embodiments, the method comprises: (a) obtaining a biological sample from a human subject; and (b) detecting whether or not there is a nucleic acid sequence encoding a Gc globulin protein having the sequence of SEQ ID NO: 2, 4, 6, 8, 9, or 10, or a combination thereof. In some embodiments, the subject is a pregnant female. In other embodiments, the subject is a child of a human subject, while yet in further embodiments, the subject is a human embryo, a human fetus, or an unborn human child. In some embodiments, the method further comprises detecting whether there is an alteration in a gene locus that encodes the Gc globulin protein. In other embodiments, the detecting comprises using PCR and primers directed to SEQ ID NO: 2, 4, 6, 8, 9, or 10. In further embodiments, the detecting comprises using hybridization, amplification, or sequencing techniques to distinguish SEQ ID NOS: 2, 4, 6, 8, 9, and/or 10. In some embodiments, the detecting comprises detecting whether mRNA expression of the Gc globulin protein is increased. In further embodiments, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof. In other embodiments, the detecting comprises detecting whether levels of serum actin-complexed Gc globulin protein are increased; levels of serum actin-free Gc globulin protein are increased; the glycosylation of actin-complexed Gc globulin is increased; the glycosylation of actin-free Gc globulin is increased; the sialylation of actin-complexed Gc globulin is increased; the sialylation of actin-free Gc globulin is increased; or a combination thereof. In some embodiments, Gc globulin levels are measured by ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof. In further embodiments, the Gc globulin protein comprises Gc1s, Gc2, Gc1f, or a combination thereof. In some embodiments, the sample comprises blood, plasma, serum, cerebrospinal fluid, sputum, lacrimal secretions, semen, vaginal secretions, fetal tissue, liver tissue, amniotic fluid, cord blood, or a combination thereof. In some embodiments, the method further comprises assessing whether or not the course of autism or ASD will be severe. For example, severity can be assessed clinically by scores on the Vineland Adaptive Behavior Scale (see Adrienne Perry, Helen E Flanagan, Jennifer Dunn Geier, Nancy L Freeman. Brief report: the Vineland Adaptive Behavior Scales in young children with autism spectrum disorders at different cognitive levels. J Aut Dev Disord 2009; 39:1066-1078) or by ADOS scores (see Gotham K, Pickles A, Lord C. Standardizing ADOS scores for a measure of severity in autism spectrum disorders. J Aut Dev Disord 2009; 39:693-705). In other embodiments, the method further comprises determining whether or not the subject will benefit from supplemental vitamin D treatments. This determination can be based on clinically available and research definitions of functional vitamin D status (for example, see Holick, Curr Drug Targets. 2011 January; 12(1):4-18 and Boullata, Curr Opin Clin Nutr Metab Care. 2010 November; 13(6):677-84).

In one aspect, the invention provides for a method for detecting the presence of or a predisposition to a vitamin D deficiency-related neurodevelopmental disorder in a human subject. In some embodiments, the method comprises: (a) obtaining a biological sample from a human subject; and (b) detecting whether or not there is an alteration in the expression of a Gc globulin protein in the subject as compared to a normal subject. In some embodiments, the method comprises: (a) obtaining a biological sample from a human subject; and (b) detecting whether or not there is a nucleic acid sequence encoding a Gc globulin protein having the sequence of SEQ ID NO: 2, 4, 6, 8, 9, or 10. In some embodiments, the subject is a pregnant female. In other embodiments, the subject is a child of a human subject, while yet in further embodiments, the subject is a human embryo, a human fetus, or an unborn human child. In some embodiments, the subject is also afflicted with autism or ASD. In some embodiments, the method further comprises detecting whether there is an alteration in a gene locus that encodes the Gc globulin protein. In other embodiments, the detecting comprises using PCR and primers directed to SEQ ID NO: 2, 4, 6, 8, 9, or 10. In further embodiments, the detecting comprises using hybridization, amplification, or sequencing techniques to distinguish SEQ ID NOS: 2, 4, 6, 8, 9, and/or 10. In some embodiments, the detecting comprises detecting whether mRNA expression of the Gc globulin protein is increased. In further embodiments, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof. In other embodiments, the detecting comprises detecting whether levels of serum actin-complexed Gc globulin protein are increased; levels of serum actin-free Gc globulin protein are increased; the glycosylation of actin-complexed Gc globulin is increased; the glycosylation of actin-free Gc globulin is increased; the sialylation of actin-complexed Gc globulin is increased; the sialylation of actin-free Gc globulin is increased; or a combination thereof. In some embodiments, Gc globulin levels are measured by ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof. In further embodiments, the Gc globulin protein comprises Gc1s, Gc2, Gc1f, or a combination thereof. In some embodiments, the sample comprises blood, plasma, serum, cerebrospinal fluid, sputum, lacrimal secretions, semen, vaginal secretions, fetal tissue, liver tissue, amniotic fluid, cord blood, or a combination thereof. In some embodiments, the vitamin D deficiency-related neurodevelopmental disorder comprises an increase in volume of lateral ventricles, a decrease in brain cortex thickness, a decrease in neurite outgrowth, an increase in dentate gyrus mitotic cells, an increase in basal ganglia mitotic cells, an increase in hypothalamus mitotic cells, a decrease in calcium uptake, a decrease in dentate gyrus apoptotic cells, a decrease in basal ganglia apoptotic cells, a decrease in hypothalamus apoptotic cells, an increase in subventricular zone (SVZ) neurospheres, a decrease in levels of nerve growth factor (NGF), a decrease in levels of Glial cell-derived neurotrophic factor (GDNF), a decrease in levels of nurr1 transcription factor, a decrease in levels of p75 neurotrophin receptor (p75NTR), a decrease in levels of catechol-O-methyltransferase (COMT), a decrease in levels of neurotrophin-3 (NT-3), a decrease in levels of neurotrophin-4 (NT-4), or a combination thereof. In some embodiments, the method further comprises assessing whether or not the vitamin D deficiency-related neurodevelopmental disorder will be severe. For example, severity can be assessed using standardized severity ratings for neurodevelopmental disorders (see Vineland Adaptive Behavior Scale described in Perry et al., J Aut Dev Disord 2009; 39:1066-1078). In other embodiments, the method further comprises determining whether or not the subject will benefit from supplemental vitamin D treatments. This determination can be based on clinically available and research definitions of functional vitamin D status (for example, see Holick, Curr Drug Targets. 2011 January; 12(1):4-18 and Boullata, Curr Opin Clin Nutr Metab Care. 2010 November; 13(6):677-84).

An aspect of the invention is directed to methods for detecting the presence of or a predisposition to a vitamin D deficiency-related immune deficit in a human subject. In some embodiments, the method comprises: (a) obtaining a biological sample from a human subject; and (b) detecting whether or not there is an alteration in the expression of a Gc globulin protein in the subject as compared to a normal subject. In some embodiments, the method comprises: (a) obtaining a biological sample from a human subject; and (b) detecting whether or not there is a nucleic acid sequence encoding a Gc globulin protein having the sequence of SEQ ID NO: 2, 4, 6, 8, 9, or 10. In some embodiments, the subject is a pregnant female. In other embodiments, the subject is a child of a human subject, while yet in further embodiments, the subject is a human embryo, a human fetus, or an unborn human child. In some embodiments, the subject is also afflicted with autism or ASD. In some embodiments, the method further comprises detecting whether there is an alteration in a gene locus that encodes the Gc globulin protein. In other embodiments, the detecting comprises using PCR and primers directed to SEQ ID NO: 2, 4, 6, 8, 9, or 10. In further embodiments, the detecting comprises using hybridization, amplification, or sequencing techniques to distinguish SEQ ID NOS: 2, 4, 6, 8, 9, and/or 10. In some embodiments, the detecting comprises detecting whether mRNA expression of the Gc globulin protein is increased. In further embodiments, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof. In other embodiments, the detecting comprises detecting whether levels of serum actin-complexed Gc globulin protein are increased; levels of serum actin-free Gc globulin protein are increased; the glycosylation of actin-complexed Gc globulin is increased; the glycosylation of actin-free Gc globulin is increased; the sialylation of actin-complexed Gc globulin is increased; the sialylation of actin-free Gc globulin is increased; or a combination thereof. In some embodiments, Gc globulin levels are measured by ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof. In further embodiments, the Gc globulin protein comprises Gc1s, Gc2, Gc1f, or a combination thereof. In some embodiments, the sample comprises blood, plasma, serum, cerebrospinal fluid, sputum, lacrimal secretions, semen, vaginal secretions, fetal tissue, liver tissue, amniotic fluid, cord blood, or a combination thereof. In some embodiments, the vitamin D deficiency-related immune deficit comprises type I diabetes mellitus, multiple sclerosis, rheumatoid arthritis, systemic lupus erythematosus, inflammatory bowel disease, asthma, a bacterial infection, a viral infection, or a combination thereof. In other embodiments, the bacterial infection comprises a Mycobacterium tuberculosis infection, a Bordatella bronchi septica infection, a Pseudomonas aeruginosa infection, a Listeria monocytogenes infection, a Group B Streptococcus infection, or a combination of listed bacterial infections. In further embodiments, the viral infection comprises an influenza infection, a hepatitis C infection, an HIV-1 infection, or a combination of the listed viral infections. In some embodiments, the method further comprises assessing whether or not the vitamin D deficiency-related immune deficit will be severe. For example, severity can be assessed based on clinically available and research definitions of functional vitamin D status as well as diagnostic assays to be developed. In other embodiments, the method further comprises determining whether or not the subject will benefit from supplemental vitamin D treatments. This determination can be based on clinically available and research definitions of functional vitamin D status (for example, see Holick, Curr Drug Targets. 2011 January; 12(1):4-18 and Boullata, Curr Opin Clin Nutr Metab Care. 2010 November; 13(6):677-84).

Accordingly, in one aspect, the invention provides a method for detecting the presence of or a predisposition to autism or an autism spectrum disorder (ASD) in a human subject or a child of a human subject. In some embodiments, the method comprises: (a) obtaining a biological sample from a human subject; (b) detecting whether or not there is an alteration in the expression of a Gc globulin protein in the subject as compared to a non-autistic subject; and (c) measuring vitamin D levels in the subject. In other embodiments, the method comprises: (a) obtaining a biological sample from a human subject; (b) detecting whether or not there is a nucleic acid sequence encoding a Gc globulin protein having the sequence of SEQ ID NO: 2, 4, 6, 8, 9, or 10, or a combination thereof; and (c) measuring vitamin D levels in the subject. In some embodiments, the subject is a pregnant female. In other embodiments, the subject is a child of a human subject, while yet in further embodiments, the subject is a human embryo, a human fetus, or an unborn human child. In some embodiments, the method further comprises detecting whether there is an alteration in a gene locus that encodes the Gc globulin protein. In other embodiments, the detecting comprises using PCR and primers directed to SEQ ID NO: 2, 4, 6, 8, 9, or 10. In further embodiments, the detecting comprises using hybridization, amplification, or sequencing techniques to distinguish SEQ ID NOS: 2, 4, 6, 8, 9, and/or 10. In some embodiments, the detecting comprises detecting whether mRNA expression of the Gc globulin protein is increased. In further embodiments, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof. In other embodiments, the detecting comprises detecting whether levels of serum actin-complexed Gc globulin protein are increased; levels of serum actin-free Gc globulin protein are increased; the glycosylation of actin-complexed Gc globulin is increased; the glycosylation of actin-free Gc globulin is increased; the sialylation of actin-complexed Gc globulin is increased; the sialylation of actin-free Gc globulin is increased; or a combination thereof. In some embodiments, Gc globulin levels are measured by ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof. In further embodiments, the Gc globulin protein comprises Gc1s, Gc2, Gc1f, or a combination thereof. In some embodiments, the sample comprises blood, plasma, serum, cerebrospinal fluid, sputum, lacrimal secretions, semen, vaginal secretions, fetal tissue, liver tissue, amniotic fluid, cord blood, or a combination thereof. In some embodiments, measurements of vitamin D levels comprises measuring total and free vitamin D levels. In some embodiments, analysis of vitamin D levels (vitamin D3, 25-hydroxyvitamin D3 and/or 24,25-dihydroxyvitamin D3) can be performed by LC-MS/MS targeting vitamin D and its metabolites (Burild et al. 2014). In other embodiments, total vitamin D levels (vitamin D3, 25-hydroxyvitamin D3 and/or 24,25-dihydroxyvitamin D3) can be measured by immunoassays including ELISA using an antibody directed to vitamin D; western blot using an antibody directed to vitamin D; or bead-based immunoassays available commercially, such as from Luminex, or other related detection platforms. In some embodiments, the method further comprises assessing whether or not the course of autism or ASD will be severe. For example, severity can be assessed clinically by scores on the Vineland Adaptive Behavior Scale (see Adrienne Perry, Helen E Flanagan, Jennifer Dunn Geier, Nancy L Freeman. Brief report: the Vineland Adaptive Behavior Scales in young children with autism spectrum disorders at different cognitive levels. J Aut Dev Disord 2009; 39:1066-1078) or by ADOS scores (see Gotham K, Pickles A, Lord C. Standardizing ADOS scores for a measure of severity in autism spectrum disorders. J Aut Dev Disord 2009; 39:693-705). In other embodiments, the method further comprises determining whether or not the subject will benefit from supplemental vitamin D treatments. This determination can be based on clinically available and research definitions of functional vitamin D status (for example, see Holick, Curr Drug Targets. 2011 January; 12(1):4-18 and Boullata, Curr Opin Clin Nutr Metab Care. 2010 November; 13(6):677-84).

In one aspect, the invention provides for a method for detecting the presence of or a predisposition to a vitamin D deficiency-related neurodevelopmental disorder in a human subject. In some embodiments, the method comprises: (a) obtaining a biological sample from a human subject; (b) detecting whether or not there is an alteration in the expression of a Gc globulin protein in the subject as compared to a non-autistic subject; and (c) measuring vitamin D levels in the subject. In other embodiments, the method comprises: (a) obtaining a biological sample from a human subject; (b) detecting whether or not there is a nucleic acid sequence encoding a Gc globulin protein having the sequence of SEQ ID NO: 2, 4, 6, 8, 9, or 10, or a combination thereof; and (c) measuring vitamin D levels in the subject.

An aspect of the invention is directed to methods for detecting the presence of or a predisposition to a vitamin D deficiency-related immune deficit in a human subject. In some embodiments, the method comprises: (a) obtaining a biological sample from a human subject; (b) detecting whether or not there is an alteration in the expression of a Gc globulin protein in the subject as compared to a non-autistic subject; and (c) measuring vitamin D levels in the subject. In other embodiments, the method comprises: (a) obtaining a biological sample from a human subject; (b) detecting whether or not there is a nucleic acid sequence encoding a Gc globulin protein having the sequence of SEQ ID NO: 2, 4, 6, 8, 9, or 10, or a combination thereof; and (c) measuring vitamin D levels in the subject.

An aspect of the invention provides for methods for treating or preventing autism or an ASD in a subject in need thereof, the method comprising administering to the subject a therapeutic amount of a pharmaceutical composition comprising a Gc globulin modulating compound, thereby treating or preventing autism or an ASD. In some embodiments, the subject is a pregnant female. In other embodiments, the subject is a child of a human subject. In further embodiments, the subject is a human embryo, a human fetus, or an unborn human child. In some embodiments, the method further comprises administering vitamin D to the subject in need. In further embodiments, administering vitamin D is conducted simultaneously with the administering of the Gc globulin modulating compound. In other embodiments, administering vitamin D is conducted sequentially in any order with the administering of the Gc globulin modulating compound. In some embodiments, the compound comprises an antibody that specifically binds to a Gc globulin protein or a fragment thereof; an antisense RNA or antisense DNA that decreases expression of a Gc globulin polypeptide; a siRNA that specifically targets a Gc globulin gene; or a combination thereof. In some embodiments, the Gc globulin protein comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof. In other embodiments, the Gc globulin protein comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof. In other embodiments, the compound comprises a peptide comprising at least about 10 amino acids of SEQ ID NO: 1, 3, 5, 11, 12, or 13 or a vector comprising a nucleic acid sequence comprising SEQ ID NO: 2, 4, 6, 8, 9, or 10. In further embodiments, administering comprises a subcutaneous, intra-muscular, intra-peritoneal, or intravenous injection; an infusion; oral, nasal, or topical delivery; or a combination thereof. In some embodiments, the Gc globulin comprises Gc1s, Gc2, Gc1f, or a combination thereof. In some embodiments, vitamin D is 25-hydroxy-vitamin D, 1,25-dihydroxyvitamin D3, cholecalciferol, or a vitamin D analogue. In other embodiments, 1,25-dihydroxyvitamin D3 is calcitriol. In further embodiments, calcitriol is administered at a concentration of at least about 25 ng, at least about 50 ng, at least about 100 ng, at least about 125 ng, at least about 150 ng, at least about 200 ng, at least about 250 ng, at least about 300 ng, at least about 350 ng, at least about 400 ng, 450 ng, at least about 500 ng, at least about 550 ng, at least about 600 ng, at least about 650 ng, at least about 700 ng, at least about 750 ng, at least about 800 ng, 850 ng, at least about 900 ng, at least about 1000 ng, at least about 1100 ng, at least about 1200 ng, at least about 1300 ng, at least about 1400 ng, or at least about 1500 ng. In other embodiments, calcitriol is administered 1 time per week, 2 times per week, 3 times per week, 4 times per week, or 5 times per week. In further embodiments, calcitriol is adjusted weekly in 250 ng increments. In some embodiments, cholecalciferol is administered at a concentration of at least about 1000 IU, at least about 2000 IU, at least about 2500 IU, at least about 3000 IU, at least about 3500 IU, at least about 4000 IU, at least about 4500 IU, at least about 5000 IU, at least about 5500 IU, at least about 6000 IU, at least about 6500 IU, at least about 7000 IU, at least about 7500 IU, at least about 8000 IU, at least about 8500 IU, at least about 9000 IU, at least about 9500 IU, at least about 10000 IU, at least about 11000 IU, at least about 12000 IU, at least about 13000 IU, at least about 14000 IU, at least about 15000 IU, at least about 16000 IU, at least about 17000 IU, at least about 18000 IU, at least about 19000 IU, at least about 20000 IU, or at least about 25000 IU. In other embodiments, cholecalciferol is administered daily. In some embodiments, the vitamin D analogue is ergo-cholecalciferol. In further embodiments, ergo-cholecalciferol is administered at a concentration of at least about 50000 IU. In other embodiments, ergo-cholecalciferol is administered weekly. In some embodiments, the method further comprises exposing the subject in need to ultraviolet light to synthesize vitamin D. In other embodiments, the subject is exposed to UV light for sessions of at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 16 minutes, at least 18 minutes, at least 20 minutes, or at least 25 minutes. In further embodiments, the subject attends a session at least once a week, at least 2 times a week, at least 3 times per week, at least 4 times per week, or at least 5 times per week. In other embodiments, the subject is exposed to a UV light session for at least 3 weeks, at least 4 weeks, at least 5 weeks, at least 6 weeks, at least 8 weeks, at least 12 weeks, or at least 16 weeks. In some embodiments, the method further comprises monitoring the autism or ASD subject during treatment with the Gc globulin, alone or in combination with vitamin D. In some embodiments, an improvement in the ability to establish friendships with children the same age, having empathy, and the ability to develop nonverbal communicative skills (for example, eye-to-eye gazing, facial expressions, and body posture) will be assessed. In some embodiments, improvements in learning to talk, improvements in the ability to talk, improvements in the ability to initiate or maintain a conversation, improvements in the ability to interpret or understand implied meaning of words, as well as a decrease in the repetitive use of language will be assessed during treatment.

An aspect of the invention provides for methods for increasing free serum vitamin D levels of a subject in need thereof, the method comprising: administering to the subject a therapeutic amount of a pharmaceutical composition comprising a Gc globulin modulating compound, thereby altering the proportion of vitamin D that is bound to Gc globlin, and increasing the levels of free serum vitamin D. In some embodiments, the subject is afflicted with autism or an autism spectrum disorder. In other embodiments, the subject is afflicted with a vitamin D deficiency-related neurodevelopmental disorder. In some embodiments, the vitamin D deficiency-related neurodevelopmental disorder comprises an increase in volume of lateral ventricles, a decrease in brain cortex thickness, a decrease in neurite outgrowth, an increase in dentate gyrus mitotic cells, an increase in basal ganglia mitotic cells, an increase in hypothalamus mitotic cells, a decrease in calcium uptake, a decrease in dentate gyrus apoptotic cells, a decrease in basal ganglia apoptotic cells, a decrease in hypothalamus apoptotic cells, an increase in subventricular zone (SVZ) neurospheres, a decrease in levels of nerve growth factor (NGF), a decrease in levels of Glial cell-derived neurotrophic factor (GDNF), a decrease in levels of nurr1 transcription factor, a decrease in levels of p75 neurotrophin receptor (p75NTR), a decrease in levels of catechol-O-methyltransferase (COMT), a decrease in levels of neurotrophin-3 (NT-3), a decrease in levels of neurotrophin-4 (NT-4), or a combination thereof. In further embodiments, the subject is afflicted with a vitamin D deficiency-related immune deficit. In some embodiments, the vitamin D deficiency-related immune deficit comprises type I diabetes mellitus, multiple sclerosis, rheumatoid arthritis, systemic lupus erythematosus, inflammatory bowel disease, asthma, a bacterial infection, a viral infection, or a combination thereof. In other embodiments, the bacterial infection comprises a Mycobacterium tuberculosis infection, a Bordatella bronchi septica infection, a Pseudomonas aeruginosa infection, a Listeria monocytogenes infection, a Group B Streptococcus infection, or a combination of listed bacterial infections. In further embodiments, the viral infection comprises an influenza infection, a hepatitis C infection, an HIV-1 infection, or a combination of the listed viral infections. In some embodiments, the subject is a pregnant female. In other embodiments, the subject is a child of a human subject. In further embodiments, the subject is a human embryo, a human fetus, or an unborn human child. In some embodiments, the method further comprises administering vitamin D to the subject in need. In further embodiments, administering vitamin D is conducted simultaneously with the administering of the Gc globulin modulating compound. In other embodiments, administering vitamin D is conducted sequentially in any order with the administering of the Gc globulin modulating compound. In some embodiments, the compound comprises an antibody that specifically binds to a Gc globulin protein or a fragment thereof; an antisense RNA or antisense DNA that decreases expression of a Gc globulin polypeptide; a siRNA that specifically targets a Gc globulin gene; or a combination thereof. In some embodiments, the Gc globulin protein comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof. In other embodiments, the Gc globulin protein comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof. In other embodiments, the compound comprises a peptide comprising at least about 10 amino acids of SEQ ID NO: 1, 3, 5, 11, 12, or 13 or a vector comprising a nucleic acid sequence comprising SEQ ID NO: 2, 4, 6, 8, 9, or 10. In further embodiments, administering comprises a subcutaneous, intra-muscular, intra-peritoneal, or intravenous injection; an infusion; oral, nasal, or topical delivery; or a combination thereof. In some embodiments, the Gc globulin comprises Gc1s, Gc2, Gc1f, or a combination thereof. In some embodiments, vitamin D is 25-hydroxy-vitamin D, 1,25-dihydroxyvitamin D3, cholecalciferol, or a vitamin D analogue. In other embodiments, 1,25-dihydroxyvitamin D3 is calcitriol. In further embodiments, calcitriol is administered at a concentration of at least about 25 ng, at least about 50 ng, at least about 100 ng, at least about 125 ng, at least about 150 ng, at least about 200 ng, at least about 250 ng, at least about 300 ng, at least about 350 ng, at least about 400 ng, 450 ng, at least about 500 ng, at least about 550 ng, at least about 600 ng, at least about 650 ng, at least about 700 ng, at least about 750 ng, at least about 800 ng, 850 ng, at least about 900 ng, at least about 1000 ng, at least about 1100 ng, at least about 1200 ng, at least about 1300 ng, at least about 1400 ng, or at least about 1500 ng. In other embodiments, calcitriol is administered 1 time per week, 2 times per week, 3 times per week, 4 times per week, or 5 times per week. In further embodiments, calcitriol is adjusted weekly in 250 ng increments. In some embodiments, cholecalciferol is administered at a concentration of at least about 1000 IU, at least about 2000 IU, at least about 2500 IU, at least about 3000 IU, at least about 3500 IU, at least about 4000 IU, at least about 4500 IU, at least about 5000 IU, at least about 5500 IU, at least about 6000 IU, at least about 6500 IU, at least about 7000 IU, at least about 7500 IU, at least about 8000 IU, at least about 8500 IU, at least about 9000 IU, at least about 9500 IU, at least about 10000 IU, at least about 11000 IU, at least about 12000 IU, at least about 13000 IU, at least about 14000 IU, at least about 15000 IU, at least about 16000 IU, at least about 17000 IU, at least about 18000 IU, at least about 19000 IU, at least about 20000 IU, or at least about 25000 IU. In other embodiments, cholecalciferol is administered daily. In some embodiments, the vitamin D analogue is ergo-cholecalciferol. In further embodiments, ergo-cholecalciferol is administered at a concentration of at least about 50000 IU. In other embodiments, ergo-cholecalciferol is administered weekly. In some embodiments, the method further comprises exposing the subject in need to ultraviolet light to synthesize vitamin D. In other embodiments, the subject is exposed to UV light for sessions of at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 16 minutes, at least 18 minutes, at least 20 minutes, or at least 25 minutes. In further embodiments, the subject attends a session at least once a week, at least 2 times a week, at least 3 times per week, at least 4 times per week, or at least 5 times per week. In other embodiments, the subject is exposed to a UV light session for at least 3 weeks, at least 4 weeks, at least 5 weeks, at least 6 weeks, at least 8 weeks, at least 12 weeks, or at least 16 weeks.

An aspect of the invention provides for compositions for elevating serum vitamin D levels in a subject, the composition in an admixture of a pharmaceutically acceptable carrier comprising a Gc globulin modulating compound. In some embodiments, the pharmaceutically acceptable carrier comprises water, a glycol, an ester, an alcohol, a lipid, or a combination thereof. In other embodiments, the compound comprises an antibody that specifically binds to a Gc globulin protein or a fragment thereof; an antisense RNA or antisense DNA that decreases expression of a Gc globulin polypeptide; a siRNA that specifically targets a Gc globulin gene; or a combination thereof. In further embodiments, the compound comprises a peptide comprising at least about 10 amino acids of SEQ ID NO: 1, 3, 5, 11, 12, or 13 or a vector comprising a nucleic acid sequence comprising SEQ ID NO: 2, 4, 6, 8, 9, or 10. In some embodiments, the Gc globulin protein comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof.

An aspect of the inventions provides for diagnostic kits for determining whether a sample from a subject exhibits a presence of or a predisposition to autism or an autism spectrum disorder (ASD). In some embodiments, the kit comprises a nucleic acid primer that specifically hybridizes to a Gc globulin biomarker comprising SEQ ID NO: 2, 4, 6, 8, 9, or 10, wherein the PCR primer will prime a polymerase reaction only when a Gc globulin biomarker is present. In other embodiments, the kit provides for a set of nucleic acid primers that specifically hybridize to a Gc globulin biomarker comprising SEQ ID NO: 2, 4, 6, 8, 9, or 10. In some embodiments, the kit comprises an antibody that specifically binds to a Gc globulin biomarker comprising SEQ ID NO: 1, 3, 5, 11, 12, or 13, wherein the antibody will recognize the protein only when a Gc globulin biomarker is present. In other embodiments, the Gc globulin biomarker is Gc1s, Gc2, Gc1f, or a combination thereof. In further embodiments, the sample is from a human. In some embodiments, the sample comprises cord blood, blood, skin, plasma, serum, cerebrospinal fluid, or a combination thereof.

An aspect of the invention provides for methods for treating or preventing autism or an ASD in a subject in need thereof, where the method comprises administering to the subject a therapeutic amount of GcMAF, thereby treating or preventing autism or an ASD. In some embodiments, the amount of GcMAF is at least about 0.0001 at least about 0.00025 at least about 0.0005 at least about 0.00075 at least about 0.001 at least about 0.0025 at least about 0.005 at least about 0.0075 at least about 0.01 at least about 0.025 at least about 0.05 at least about 0.075 at least about 0.1 at least about 0.25 at least about 0.5 μg, at least about 0.75 μg, at least about 1 μg, at least about 5 μg, or at least about 10 μg. In some embodiments, the subject is a pregnant female. In other embodiments, the subject is a child of a human subject. In further embodiments, the subject is a human embryo, a human fetus, or an unborn human child. In some embodiments, the administering comprises a subcutaneous, intra-muscular, intra-peritoneal, or intravenous injection; an infusion; oral, nasal, or topical delivery; or a combination thereof. In other embodiments, the method further comprises administering vitamin D to the subject in need. In some embodiments, the vitamin D is 25-hydroxy-vitamin D, 1,25-dihydroxyvitamin D3, cholecalciferol, or a vitamin D analogue. In other embodiments, 1,25-dihydroxyvitamin D3 is calcitriol. In some embodiments, calcitriol is administered at a concentration of at least about 25 ng, at least about 50 ng, at least about 100 ng, at least about 125 ng, at least about 150 ng, at least about 200 ng, at least about 250 ng, at least about 300 ng, at least about 350 ng, at least about 400 ng, 450 ng, at least about 500 ng, at least about 550 ng, at least about 600 ng, at least about 650 ng, at least about 700 ng, at least about 750 ng, at least about 800 ng, 850 ng, at least about 900 ng, at least about 1000 ng, at least about 1100 ng, at least about 1200 ng, at least about 1300 ng, at least about 1400 ng, or at least about 1500 ng. In further embodiments, calcitriol is administered 1 time per week, 2 times per week, 3 times per week, 4 times per week, or 5 times per week. In other embodiments, calcitriol is adjusted weekly in 250 ng increments. In some embodiments, cholecalciferol is administered at a concentration of at least about 1000 IU, at least about 2000 IU, at least about 2500 IU, at least about 3000 IU, at least about 3500 IU, at least about 4000 IU, at least about 4500 IU, at least about 5000 IU, at least about 5500 IU, at least about 6000 IU, at least about 6500 IU, at least about 7000 IU, at least about 7500 IU, at least about 8000 IU, at least about 8500 IU, at least about 9000 IU, at least about 9500 IU, at least about 10000 IU, at least about 11000 IU, at least about 12000 IU, at least about 13000 IU, at least about 14000 IU, at least about 15000 IU, at least about 16000 IU, at least about 17000 IU, at least about 18000 IU, at least about 19000 IU, at least about 20000 IU, or at least about 25000 IU. In other embodiments, cholecalciferol is administered daily. In some embodiments, the vitamin D analogue is ergo-cholecalciferol. In further embodiments, ergo-cholecalciferol is administered at a concentration of at least about 50000 IU. In other embodiments, ergo-cholecalciferol is administered weekly. In some embodiments, the method further comprises exposing the subject in need to ultraviolet light to synthesize vitamin D. In other embodiments, the subject is exposed to UV light for sessions of at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 16 minutes, at least 18 minutes, at least 20 minutes, or at least 25 minutes. In further embodiments, the subject attends a session at least once a week, at least 2 times a week, at least 3 times per week, at least 4 times per week, or at least 5 times per week. In yet other embodiments, the subject is exposed to a UV light session for at least 3 weeks, at least 4 weeks, at least 5 weeks, at least 6 weeks, at least 8 weeks, at least 12 weeks, or at least 16 weeks.

An aspect of the invention provides for methods for treating or preventing a vitamin D deficiency-related neurodevelopmental disorder in a subject in need thereof, where the method comprises administering to the subject a therapeutic amount of GcMAF, thereby treating or preventing the vitamin D deficiency-related neurodevelopemental disorder. In some embodiments, the amount of GcMAF is at least about 0.0001 at least about 0.00025 at least about 0.0005 at least about 0.00075 at least about 0.001 at least about 0.0025 at least about 0.005 at least about 0.0075 at least about 0.01 at least about 0.025 at least about 0.05 at least about 0.075 at least about 0.1 at least about 0.25 at least about 0.5 at least about 0.75 at least about 1 at least about 5 or at least about 10 μg. In some embodiments, the subject is a pregnant female. In other embodiments, the subject is a child of a human subject. In further embodiments, the subject is a human embryo, a human fetus, or an unborn human child. In some embodiments, the administering comprises a subcutaneous, intra-muscular, intra-peritoneal, or intravenous injection; an infusion; oral, nasal, or topical delivery; or a combination thereof. In other embodiments, the method further comprises administering vitamin D to the subject in need. In some embodiments, the vitamin D is 25-hydroxy-vitamin D, 1,25-dihydroxyvitamin D3, cholecalciferol, or a vitamin D analogue. In other embodiments, 1,25-dihydroxyvitamin D3 is calcitriol. In some embodiments, calcitriol is administered at a concentration of at least about 25 ng, at least about 50 ng, at least about 100 ng, at least about 125 ng, at least about 150 ng, at least about 200 ng, at least about 250 ng, at least about 300 ng, at least about 350 ng, at least about 400 ng, 450 ng, at least about 500 ng, at least about 550 ng, at least about 600 ng, at least about 650 ng, at least about 700 ng, at least about 750 ng, at least about 800 ng, 850 ng, at least about 900 ng, at least about 1000 ng, at least about 1100 ng, at least about 1200 ng, at least about 1300 ng, at least about 1400 ng, or at least about 1500 ng. In further embodiments, calcitriol is administered 1 time per week, 2 times per week, 3 times per week, 4 times per week, or 5 times per week. In other embodiments, calcitriol is adjusted weekly in 250 ng increments. In some embodiments, cholecalciferol is administered at a concentration of at least about 1000 IU, at least about 2000 IU, at least about 2500 IU, at least about 3000 IU, at least about 3500 IU, at least about 4000 IU, at least about 4500 IU, at least about 5000 IU, at least about 5500 IU, at least about 6000 IU, at least about 6500 IU, at least about 7000 IU, at least about 7500 IU, at least about 8000 IU, at least about 8500 IU, at least about 9000 IU, at least about 9500 IU, at least about 10000 IU, at least about 11000 IU, at least about 12000 IU, at least about 13000 IU, at least about 14000 IU, at least about 15000 IU, at least about 16000 IU, at least about 17000 IU, at least about 18000 IU, at least about 19000 IU, at least about 20000 IU, or at least about 25000 IU. In other embodiments, cholecalciferol is administered daily. In some embodiments, the vitamin D analogue is ergo-cholecalciferol. In further embodiments, ergo-cholecalciferol is administered at a concentration of at least about 50000 IU. In other embodiments, ergo-cholecalciferol is administered weekly. In some embodiments, the method further comprises exposing the subject in need to ultraviolet light to synthesize vitamin D. In other embodiments, the subject is exposed to UV light for sessions of at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 16 minutes, at least 18 minutes, at least 20 minutes, or at least 25 minutes. In further embodiments, the subject attends a session at least once a week, at least 2 times a week, at least 3 times per week, at least 4 times per week, or at least 5 times per week. In yet other embodiments, the subject is exposed to a UV light session for at least 3 weeks, at least 4 weeks, at least 5 weeks, at least 6 weeks, at least 8 weeks, at least 12 weeks, or at least 16 weeks. In some embodiments, the vitamin D deficiency-related neurodevelopmental disorder comprises an increase in volume of lateral ventricles, a decrease in brain cortex thickness, a decrease in neurite outgrowth, an increase in dentate gyrus mitotic cells, an increase in basal ganglia mitotic cells, an increase in hypothalamus mitotic cells, a decrease in calcium uptake, a decrease in dentate gyrus apoptotic cells, a decrease in basal ganglia apoptotic cells, a decrease in hypothalamus apoptotic cells, an increase in subventricular zone (SVZ) neurospheres, a decrease in levels of nerve growth factor (NGF), a decrease in levels of Glial cell-derived neurotrophic factor (GDNF), a decrease in levels of nurr1 transcription factor, a decrease in levels of p75 neurotrophin receptor (p75NTR), a decrease in levels of catechol-O-methyltransferase (COMT), a decrease in levels of neurotrophin-3 (NT-3), a decrease in levels of neurotrophin-4 (NT-4), or a combination thereof.

An aspect of the invention provides for methods for treating or preventing a vitamin D deficiency-related immune deficit in a subject in need thereof, where the method comprises administering to the subject a therapeutic amount of GcMAF, thereby treating or preventing the vitamin D deficiency-related immune deficit. In some embodiments, the amount of GcMAF is at least about 0.0001 at least about 0.00025 at least about 0.0005 at least about 0.00075 at least about 0.001 at least about 0.0025 at least about 0.005 at least about 0.0075 at least about 0.01 at least about 0.025 at least about 0.05 at least about 0.075 at least about 0.1 at least about 0.25 at least about 0.5 at least about 0.75 at least about 1 at least about 5 or at least about 10 μg. In some embodiments, the subject is a pregnant female. In other embodiments, the subject is a child of a human subject. In further embodiments, the subject is a human embryo, a human fetus, or an unborn human child. In some embodiments, the administering comprises a subcutaneous, intra-muscular, intra-peritoneal, or intravenous injection; an infusion; oral, nasal, or topical delivery; or a combination thereof. In other embodiments, the method further comprises administering vitamin D to the subject in need. In some embodiments, the vitamin D is 25-hydroxy-vitamin D, 1,25-dihydroxyvitamin D3, cholecalciferol, or a vitamin D analogue. In other embodiments, 1,25-dihydroxyvitamin D3 is calcitriol. In some embodiments, calcitriol is administered at a concentration of at least about 25 ng, at least about 50 ng, at least about 100 ng, at least about 125 ng, at least about 150 ng, at least about 200 ng, at least about 250 ng, at least about 300 ng, at least about 350 ng, at least about 400 ng, 450 ng, at least about 500 ng, at least about 550 ng, at least about 600 ng, at least about 650 ng, at least about 700 ng, at least about 750 ng, at least about 800 ng, 850 ng, at least about 900 ng, at least about 1000 ng, at least about 1100 ng, at least about 1200 ng, at least about 1300 ng, at least about 1400 ng, or at least about 1500 ng. In further embodiments, calcitriol is administered 1 time per week, 2 times per week, 3 times per week, 4 times per week, or 5 times per week. In other embodiments, calcitriol is adjusted weekly in 250 ng increments. In some embodiments, cholecalciferol is administered at a concentration of at least about 1000 IU, at least about 2000 IU, at least about 2500 IU, at least about 3000 IU, at least about 3500 IU, at least about 4000 IU, at least about 4500 IU, at least about 5000 IU, at least about 5500 IU, at least about 6000 IU, at least about 6500 IU, at least about 7000 IU, at least about 7500 IU, at least about 8000 IU, at least about 8500 IU, at least about 9000 IU, at least about 9500 IU, at least about 10000 IU, at least about 11000 IU, at least about 12000 IU, at least about 13000 IU, at least about 14000 IU, at least about 15000 IU, at least about 16000 IU, at least about 17000 IU, at least about 18000 IU, at least about 19000 IU, at least about 20000 IU, or at least about 25000 IU. In other embodiments, cholecalciferol is administered daily. In some embodiments, the vitamin D analogue is ergo-cholecalciferol. In further embodiments, ergo-cholecalciferol is administered at a concentration of at least about 50000 IU. In other embodiments, ergo-cholecalciferol is administered weekly. In some embodiments, the method further comprises exposing the subject in need to ultraviolet light to synthesize vitamin D. In other embodiments, the subject is exposed to UV light for sessions of at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 16 minutes, at least 18 minutes, at least 20 minutes, or at least 25 minutes. In further embodiments, the subject attends a session at least once a week, at least 2 times a week, at least 3 times per week, at least 4 times per week, or at least 5 times per week. In yet other embodiments, the subject is exposed to a UV light session for at least 3 weeks, at least 4 weeks, at least 5 weeks, at least 6 weeks, at least 8 weeks, at least 12 weeks, or at least 16 weeks. In some embodiments, the vitamin D deficiency-related immune deficit comprises type I diabetes mellitus, multiple sclerosis, rheumatoid arthritis, systemic lupus erythematosus, inflammatory bowel disease, asthma, a bacterial infection, a viral infection, or a combination thereof. In other embodiments, the bacterial infection comprises a Mycobacterium tuberculosis infection, a Bordatella bronchi septica infection, a Pseudomonas aeruginosa infection, a Listeria monocytogenes infection, a Group B Streptococcus infection, or a combination thereof. In further embodiments, the viral infection comprises an influenza infection, a hepatitis C infection, an HIV-1 infection, or a combination thereof.

### BRIEF DESCRIPTION OF THE FIGURES

The patent or application file contains at least one drawing executed in color.

FIG. 1 is a graph showing the ASD/Control and Control/Control ratios for all of the peptides of the proteins indicated.

FIG. 2 is a bar graph showing ASD/Control ratios in male (SEQ ID NOS 7, 7, 7, 19, 19, 19, 20, 20, 20, 21, 21, 21, 22, 22, 22, 23, 23 and 23, respectively, in order of appearance) (top graph) and female (SEQ ID NOS 7, 7, 7, 19, 19, 19, 20, 20, 20, 21, 21, 21, 22, 22, 22, 23, 23 and 23, respectively, in order of appearance) (bottom graph) subjects. FIG. 2 also discloses the sequences “DATPT” as SEQ ID NO: 24, “EATPT” as SEQ ID NO: 25 and “DATPK” as SEQ ID NO: 26.

FIG. 3 shows a nucleotide alignment (positions 1261-1350) of the primary Gc alleles, Gc1f (SEQ ID NO: 8), Gc1s (SEQ ID NO: 9), Gc2 (SEQ ID NO: 10) (ref|NM_001204306.11). Underlining with bold text at nucleotide position 1296 of the coding sequence indicates site of a T→G change (Gc1s). Underlining with bold text at nucleotide position 1307 of the coding sequence indicates site of a C→A change (Gc2). Together, the identity of the nucleotides at these two sites allow the three Gc variants to be distinguished.

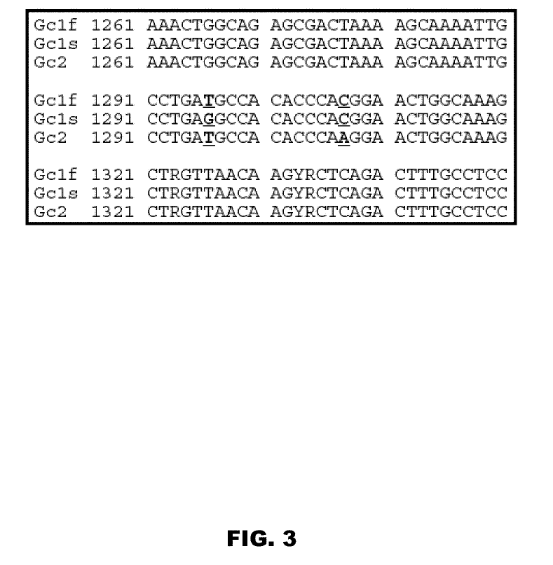

FIG. 4 shows an amino acid alignment of the major Gc variants, Gc1f (SEQ ID NO: 11), Gc1s (SEQ ID NO: 12), Gc2 (SEQ ID NO: 13) (ref|NM_001204306.11). Underlining and bold text at amino acid position 432 indicates the site of a D→E (aspartic acid→glutamic acid) amino acid change (Gc1s). Underlining and bold text at amino acid position 436 indicates the site of a T→K (threonine→lysine) amino acid change (Gc2). Together, the identity of the amino acides at these two sites allow the three major Gc variants, Gc1f, Gc1s, and Gc2, to be distinguished.

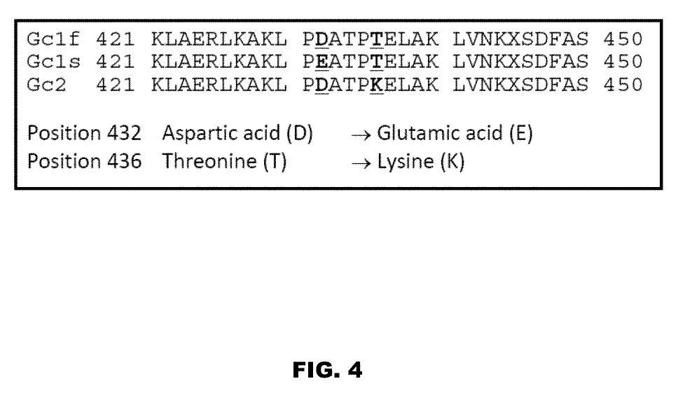

FIG. 5 shows the distribution of Gc variants in ASD and control females and males. FIG. 5 discloses the sequences “AKLPDATPK” as SEQ ID NO: 22, “LPDATPTELAK” as SEQ ID NO: 19 and “LPEATPTELAK” as SEQ ID NO: 21.

FIG. 6 shows Gc globulin polymorphisms and free vitamin D. FIG. 6 discloses the sequences “DATPT” as SEQ ID NO: 24, “EATPT” as SEQ ID NO: 25 and “DATPK” as SEQ ID NO: 26.

### DETAILED DESCRIPTION OF THE INVENTION

Autism, one of the ASDs, is mostly diagnosed clinically using behavioral criteria because few specific biological markers are known for diagnosing the disease. Autism spectrum disorders (ASD) are defined by impairments in verbal and non-verbal communication, social interactions, and repetitive and stereotyped behaviors (DSM-IV-TR criteria, American Psychiatric Association, 2000). Autism is a neuropsychiatric developmental disorder characterized by impaired verbal communication, non-verbal communication, and reciprocal social interaction. It is also characterized by restricted and stereotyped patterns of interests and activities, as well as the presence of developmental abnormalities by 3 years of age (Bailey et al., (1996) J Child Psychol Psychiatry 37(1):89-126). Autism-associated disorders, diseases or pathologies can comprise any metabolic, immune or systemic disorders; gastrointestinal disorders; epilepsy; congenital malformations or genetic syndromes; anxiety, depression, or AD/HD; or speech delay and motor in-coordination.

The prevalence of autism in the US is about 1 in 91 births and, largely due to changes in diagnostic practices, services, and public awareness. Autism is growing at the fastest pace of any developmental disability (10-17%) (Fombonne, E. (2003). The prevalence of autism. JAMA 289(1): 87-9). Care and treatment of autism costs the U.S. healthcare system $90B annually. Early detection and intervention can result in reducing life-long costs. In the last 5 years, federal funding for autism research rose by 16.1%. The Autism Society is currently lobbying Congress for $37 million for autism monitoring and studies, another $16.5 million for autism screening and academic research. At present, few tools outside psychiatric evaluation are available for diagnosing autism.

The present invention provides the discovery and the identification of Gc globulins (Gc), e.g., GcF1, Gc1s, and/or Gc2, as biomarkers for human Autism Spectrum Disorders. Gc1F, for example, is a biomarker that is detectable in umbilical cord blood plasma and can be tested for at birth. Gc1F has a higher affinity for vitamin D than other Gc variants, and its presence is therefore associated with lower levels of free 25-hydroxy-vitamin D levels in the bloodstream. As discussed herein, elevated levels of Gc globulin in the umbilical cord plasma of ASD patients were observed relative to control cases. This increase is mainly accounted for by a particular variant of Gc called Gc1F, which has increased affinity for vitamin D. Without being bound by theory, increased Gc1F levels result in reduced levels of free vitamin D, and can be a causal factor for ASD. The present invention provides for methods to use genes encoding Gc globulin molecules and corresponding expression products for the diagnosis, prevention and treatment of autism and autism spectrum disorders. The methods of the invention are useful in various subjects, such as humans, including adults (e.g., pregnant females), children, and developing human fetuses at the prenatal stage.

The practice of aspects of the present invention can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3^{rd} Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). All patents, patent applications and references cited herein are incorporated by reference in their entireties.

The Gc globulin gene locus can comprise all Gc globulin sequences or products in a cell or organism, including Gc globulin coding sequences, Gc globulin non-coding sequences (e.g., introns), and Gc globulin regulatory sequences controlling transcription and/or translation (e.g., promoter, enhancer, terminator).

Gc globulin is a vitamin D binding protein. It is encoded by the Gc gene, located at chromosome 4q11-q13. Gc globulin is a multifunctional, highly expressed, polymorphic serum protein that is 458 amino acids. Sequence variations at codons 416 and 420 in exon 11 of the Gc gene give rise to three Gc variants: Gc1 fast migrating (Gc1F), Gc1 slow migrating (Gc1S) and Gc2 (Leandro, et al., Brazilian Journal of Medical and Biological Research (2009) 42: 312-322; Ye et al., Metabolism (2001) 50(3):366-9). These variants differ in their amino-acid sequences, as well as by glycosylation. The Gc1F variant arises from the ancestral Gc1F allele. The two other variants, Gc1S and Gc2, each contain a single amino acid change compared to the Gc1F sequence: (a) D416E for Gc1S; and (b) T420K for Gc2. Combinations of the three Gc variants result in six common circulating phenotypes: Gc1F/Gc1F, Gc1F/Gc1S, Gc1S/Gc1S, Gc1F/Gc2, Gc1S/Gc2, and Gc2/Gc2 (Leandro, et al., Brazilian Journal of Medical and Biological Research (2009) 42: 312-322; Lauridsen et al., Calcif Tissue Int. (2005) 77(1):15-22).

In the context of the invention, the Gc gobulin gene also encompasses its variants, analogs and fragments thereof, including alleles thereof (e.g., germline mutations) which are related to susceptibility to autism and/or autism spectrum disorders.

As used herein, “vitamin D binding activity” means the ability of a polypeptide to bind a vitamin D, such as 25-hydroxy-vitamin D, colecalciferol, or a vitamin D analogue (e.g., ergo-colecalciferol), and subsequently facilitate uptake of vitamin D from the serum or extracellular millieu into a cell (e.g., a liver cell, a kidney cell). Vitamin D binding can be measured according to the methods described by Heijboer et al., Clin Chem. 2012 Jan. 12 (PMID: 22247500). In one embodiment, vitamin D binding activity can be measured by determining vitamin D binding and uptake as described in Heijboer et al., as well as the ability to increase extracellular or serum vitamin D levels. Non-limiting examples of a vitamin D binding protein include Gc, Gc1F, Gc1s, and Gc2.

SEQ ID NO: 1 is the translation of the coding sequence common to all three reference sequences for Vitamin D Binding Protein. This is the amino acid sequence of vitamin D binding protein, based on translation of the coding sequence common to all three reference sequences (gi|324021742|ref|NM_001204306.11, transcript variant 2; gi|324021741|ref|NM_000583.31, transcript variant 1; and gi|324021744|ref|NM_001204307.11, transcript variant 3). The amino acid sequence thus represents the Gc1f variant of vitamin D binding protein as per the variant represented in each of the three reference sequences. Markers for the two non-synonymous changes distinguishing the three primary Gc variants (Gc1f, Gc1s and Gc2) are highlighted and underlined in bold text.

The bold text and grey highlighting indicate the amino acid marker at position 432 below of the non-cleaved sequence (SEQ ID NO: 1), where a D→E (aspartic acid→glutamic acid) amino acid change corresponds to Gc1s.

The underlined and bold text indicate the amino acid marker at position 436 below of the non-cleaved sequence (SEQ ID NO: 1), where a T→K (threonine→lysine) amino acid change corresponds to Gc2.

SEQ ID NO: 1 is the human amino acid sequence corresponding to the the vitamin D binding protein (GC), transcript variant 2 precursor (residues 1-474) having GenBank Accession No. NP_001191235, where the italicized font corresponds to the presequence:

| | |
|---|---|
| 1 | MKRVLVLLLA VAFGHALERG RDYEKNKVCK EFSHLGKEDF TSLSLVLYSR KFPSGTFEQV |
| 61 | SQLVKEVVSL TEACCAEGAD PDCYDTRTSA LSAKSCESNS PFPVHPGTAE CCTKEGLERK |
| 121 | LCMAALKHQP QEFPTYVEPT NDEICEAFRK DPKEYANQFM WEYSTNYGQA PLSLLVSYTK |
| 181 | SYLSMVGSCC TSASPTVCFL KERLQLKHLS LLTTLSNRVC SQYAAYGEKK SRLSNLIKLA |
| 241 | QKVPTADLED VLPLAEDITN ILSKCCESAS EDCMAKELPE HTVKLCDNLS TKNSKFEDCC |
| 301 | QEKTAMDVFV CTYFMPAAQL PELPDVELPT NKDVCDPGNT KVMDKYTFEL SRRTHLPEVF |
| 361 | LSKVLEPTLK SLGECCDVED STTCFNAKGP LLKKELSSFI DKGQELCADY SENTFTEYKK |
| 421 | 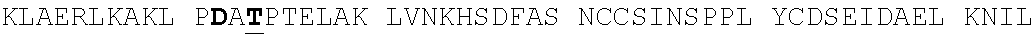 |

SEQ ID NO: 2 is the reference sequence for Vitamin D Binding Protein, non-coding and coding regions. This sequence comprises the non-coding and coding regions of the reference sequence for vitamin D binding protein, gi|324021742|ref|NM_001204306.11Homo sapiens group-specific component (vitamin D binding protein) (GC), transcript variant 2, mRNA. Reference sequence NM_001204306.1 (variant 2) differs from the two other available reference sequences, NM_000583.3 (variant 1) and NM_001204307.1 (variant 3), only in the non-coding region. All 3 reference sequences represent the Gc1f variant of vitamin D binding protein.

Markers for the two non-synonymous changes distinguishing the three primary Gc variants (Gc1f, Gc1s and Gc2) are highlighted (identifying the triplet) and underlined (identifying the nucleotide change) as defined in the key.

The bold text and grey highlighting indicates the triplet marker for the first non-synonymous change distinguishing the three major Gc variants, Gc1f, Gc1s, and Gc2. The underlining with bold text below of the non-cleaved sequence (SEQ ID NO: 2) indicates the nucleotide position 1511 of the entire reference sequence (equivalent to nucleotide position 1296 of the coding sequence) where a T→G change corresponds to Gc1s.

The grey highlighted text indicates the triplet marker for the second non-synonymous change distinguishing the three major Gc variants, Gc1f, Gc1s, and Gc2. The underlined text below of the non-cleaved sequence (SEQ ID NO: 2) indicates position 1522 of the entire reference sequence (equivalent to position 1307 of the coding sequence) where a C→A change corresponds to Gc2.

SEQ ID NO: 2 is the human nucleic acid sequence corresponding to the vitamin D binding protein (GC), transcript variant 2 precursor (bps 1-1895) having GenBank Accession No. NM_001204306. The underlined ATG and TAG correspond to the start and stop codons, respectively, and where the italicized font in the coding region corresponds to the presequence:

| | |
|---|---|
| 1 | actgctcatt cttcagccac tagtgctgga cagtccccta gcttctctta cactgcatac |
| 61 | ctgtgtctga gtactctttt catcagtcac tcagccaggg tctgcagcat gaacccagca |
| 121 | gctggtgtcc cctatgagga actcagcaac agattgcgac agaacccttg agaacaaagg |
| 181 | agagaggagg tgctgcaaga ctctctggta gaaaaatgaa gagggtcctg gtactactgc |
| 241 | ttgctgtggc atttggacat gctttagaga gaggccggga ttatgaaaag aataaagtct |
| 301 | gcaaggaatt ctcccatctg ggaaaggagg acttcacatc tctgtcacta gtcctgtaca |
| 361 | gtagaaaatt tcccagtggc acgtttgaac aggtcagcca acttgtgaag gaagttgtct |
| 421 | ccttgaccga agcctgctgt gcggaagggg ctgaccctga ctgctatgac accaggacct |
| 481 | cagcactgtc tgccaagtcc tgtgaaagta attctccatt ccccgttcac ccaggcactg |
| 541 | ctgagtgctg caccaaagag ggcctggaac gaaagctctg catggctgct ctgaaacacc |
| 601 | agccacagga attccctacc tacgtggaac ccacaaatga tgaaatctgt gaggcgttca |
| 661 | ggaaagatcc aaaggaatat gctaatcaat ttatgtggga atattccact aattacggac |
| 721 | aagctcctct gtcactttta gtcagttaca ccaagagtta tctttctatg gtagggtcct |
| 781 | gctgtacctc tgcaagccca actgtatgct ttttgaaaga gagactccag cttaaacatt |
| 841 | tatcacttct caccactctg tcaaatagag tctgctcaca atatgctgct tatggggaga |
| 901 | agaaatcaag gctcagcaat ctcataaagt tagcccaaaa agtgcctact gctgatctgg |
| 961 | aggatgtttt gccactagct gaagatatta ctaacatcct ctccaaatgc tgtgagtctg |
| 1021 | cctctgaaga ttgcatggcc aaagagctgc ctgaacacac agtaaaactc tgtgacaatt |
| 1081 | tatccacaaa gaattctaag tttgaagact gttgtcaaga aaaaacagcc atggacgttt |
| 1141 | ttgtgtgcac ttacttcatg ccagctgccc aactccccga gcttccagat gtagagttgc |
| 1201 | ccacaaacaa agatgtgtgt gatccaggaa acaccaaagt catggataag tatacatttg |
| 1261 | aactaagcag aaggactcat cttccggaag tattcctcag taaggtactt gagccaaccc |
| 1321 | taaaaagcct tggtgaatgc tgtgatgttg aagactcaac tacctgtttt aatgctaagg |
| 1381 | gccctctact aaagaaggaa ctatcttctt tcattgacaa gggacaagaa ctatgtgcag |
| 1441 | attattcaga aaatacattt actgagtaca agaaaaaact ggcagagcga ctaaaagcaa |
| 1501 | 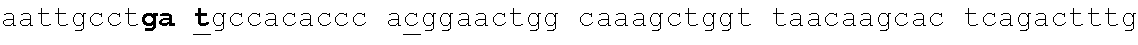 |
| 1561 | cctccaactg ctgttccata aactcacctc ctctttactg tgattcagag attgatgctg |
| 1621 | aattgaagaa tatcctgtag tcctgaagca tgtttattaa ctttgaccag agttggagcc |
| 1681 | acccagggga atgatctctg atgacctaac ctaagcaaaa ccactgagct tctgggaaga |
| 1741 | caactaggat actttctact ttttctagct acaatatctt catacaatga caagtatgat |
| 1801 | gatttgctat caaaataaat tgaaatataa tgcaaaccat aaaaaaaaaa aaaaaaaaaa |
| 1861 | aaaaaaaaaa aaaaaaaaaa aaaaaaaaaa aaaaa |

SEQ ID NO: 3 is the human amino acid sequence corresponding to the the vitamin D binding protein, Gc1s, having at position 432 a D→E (aspartic acid→glutamic acid) amino acid change, where the italicized font corresponds to the presequence:

| | |
|---|---|
| 1 | MKRVLVLLLA VAFGHALERG RDYEKNKVCK EFSHLGKEDF TSLSLVLYSR KFPSGTFEQV |
| 61 | SQLVKEVVSL TEACCAEGAD PDCYDTRTSA LSAKSCESNS PFPVHPGTAE CCTKEGLERK |
| 121 | LCMAALKHQP QEFPTYVEPT NDEICEAFRK DPKEYANQFM WEYSTNYGQA PLSLLVSYTK |
| 181 | SYLSMVGSCC TSASPTVCFL KERLQLKHLS LLTTLSNRVC SQYAAYGEKK SRLSNLIKLA |
| 241 | QKVPTADLED VLPLAEDITN ILSKCCESAS EDCMAKELPE HTVKLCDNLS TKNSKFEDCC |
| 301 | QEKTAMDVFV CTYFMPAAQL PELPDVELPT NKDVCDPGNT KVMDKYTFEL SRRTHLPEVF |
| 361 | LSKVLEPTLK SLGECCDVED STTCFNAKGP LLKKELSSFI DKGQELCADY SENTFTEYKK |
| 421 | KLAERLKAKL PEATPTELAK LVNKHSDFAS NCCSINSPPL YCDSEIDAEL KNIL |

SEQ ID NO: 4 is the human nucleic acid sequence corresponding to the the vitamin D binding protein, Gc1s, having at position 1511 a T→G nucleotide change. The underlined ATG and TAG correspond to the start and stop codons, respectively, and where the italicized font in the coding region corresponds to the presequence:

| | |
|---|---|
| 1 | actgctcatt cttcagccac tagtgctgga cagtccccta gcttctctta cactgcatac |
| 61 | ctgtgtctga gtactctttt catcagtcac tcagccaggg tctgcagcat gaacccagca |
| 121 | gctggtgtcc cctatgagga actcagcaac agattgcgac agaacccttg agaacaaagg |
| 181 | agagaggagg tgctgcaaga ctctctggta gaaaaatgaa gagggtcctg gtactactgc |
| 241 | ttgctgtggc atttggacat gctttagaga gaggccggga ttatgaaaag aataaagtct |
| 301 | gcaaggaatt ctcccatctg ggaaaggagg acttcacatc tctgtcacta gtcctgtaca |
| 361 | gtagaaaatt tcccagtggc acgtttgaac aggtcagcca acttgtgaag gaagttgtct |
| 421 | ccttgaccga agcctgctgt gcggaagggg ctgaccctga ctgctatgac accaggacct |
| 481 | cagcactgtc tgccaagtcc tgtgaaagta attctccatt ccccgttcac ccaggcactg |
| 541 | ctgagtgctg caccaaagag ggcctggaac gaaagctctg catggctgct ctgaaacacc |
| 601 | agccacagga attccctacc tacgtggaac ccacaaatga tgaaatctgt gaggcgttca |
| 661 | ggaaagatcc aaaggaatat gctaatcaat ttatgtggga atattccact aattacggac |
| 721 | aagctcctct gtcactttta gtcagttaca ccaagagtta tctttctatg gtagggtcct |
| 781 | gctgtacctc tgcaagccca actgtatgct ttttgaaaga gagactccag cttaaacatt |
| 841 | tatcacttct caccactctg tcaaatagag tctgctcaca atatgctgct tatggggaga |
| 901 | agaaatcaag gctcagcaat ctcataaagt tagcccaaaa agtgcctact gctgatctgg |
| 961 | aggatgtttt gccactagct gaagatatta ctaacatcct ctccaaatgc tgtgagtctg |
| 1021 | cctctgaaga ttgcatggcc aaagagctgc ctgaacacac agtaaaactc tgtgacaatt |
| 1081 | tatccacaaa gaattctaag tttgaagact gttgtcaaga aaaaacagcc atggacgttt |
| 1141 | ttgtgtgcac ttacttcatg ccagctgccc aactccccga gcttccagat gtagagttgc |
| 1201 | ccacaaacaa agatgtgtgt gatccaggaa acaccaaagt catggataag tatacatttg |
| 1261 | aactaagcag aaggactcat cttccggaag tattcctcag taaggtactt gagccaaccc |
| 1321 | taaaaagcct tggtgaatgc tgtgatgttg aagactcaac tacctgtttt aatgctaagg |
| 1381 | gccctctact aaagaaggaa ctatcttctt tcattgacaa gggacaagaa ctatgtgcag |
| 1441 | attattcaga aaatacattt actgagtaca agaaaaaact ggcagagcga ctaaaagcaa |
| 1501 | 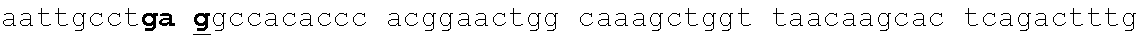 |
| 1561 | cctccaactg ctgttccata aactcacctc ctctttactg tgattcagag attgatgctg |
| 1621 | aattgaagaa tatcctgtag tcctgaagca tgtttattaa ctttgaccag agttggagcc |
| 1681 | acccagggga atgatctctg atgacctaac ctaagcaaaa ccactgagct tctgggaaga |
| 1741 | caactaggat actttctact ttttctagct acaatatctt catacaatga caagtatgat |
| 1801 | gatttgctat caaaataaat tgaaatataa tgcaaaccat aaaaaaaaaa aaaaaaaaaa |
| 1861 | aaaaaaaaaa aaaaaaaaaa aaaaaaaaaa aaaaa |

SEQ ID NO: 5 is the human amino acid sequence corresponding to the the vitamin D binding protein, Gc2, having at position 436 a T→K (threonine→lysine) amino acid change, where the italicized font corresponds to the presequence:

| | |
|---|---|
| 1 | MKRVLVLLLA VAFGHALERG RDYEKNKVCK EFSHLGKEDF TSLSLVLYSR KFPSGTFEQV |
| 61 | SQLVKEVVSL TEACCAEGAD PDCYDTRTSA LSAKSCESNS PFPVHPGTAE CCTKEGLERK |
| 121 | LCMAALKHQP QEFPTYVEPT NDEICEAFRK DPKEYANQFM WEYSTNYGQA PLSLLVSYTK |
| 181 | SYLSMVGSCC TSASPTVCFL KERLQLKHLS LLTTLSNRVC SQYAAYGEKK SRLSNLIKLA |
| 241 | QKVPTADLED VLPLAEDITN ILSKCCESAS EDCMAKELPE HTVKLCDNLS TKNSKFEDCC |
| 301 | QEKTAMDVFV CTYFMPAAQL PELPDVELPT NKDVCDPGNT KVMDKYTFEL SRRTHLPEVF |
| 361 | LSKVLEPTLK SLGECCDVED STTCFNAKGP LLKKELSSFI DKGQELCADY SENTFTEYKK |
| 421 | KLAERLKAKL PDAKPTELAK LVNKHSDFAS NCCSINSPPL YCDSEIDAEL KNIL |

SEQ ID NO: 6 is the human nucleic acid sequence corresponding to the the vitamin D binding protein, Gc2, having at position 1522 a C→A nucleotide change. The underlined ATG and TAG correspond to the start and stop codons, respectively, and where the italicized font in the coding region corresponds to the presequence:

| | |
|---|---|
| 1 | actgctcatt cttcagccac tagtgctgga cagtccccta gcttctctta cactgcatac |
| 61 | ctgtgtctga gtactctttt catcagtcac tcagccaggg tctgcagcat gaacccagca |
| 121 | gctggtgtcc cctatgagga actcagcaac agattgcgac agaacccttg agaacaaagg |
| 181 | agagaggagg tgctgcaaga ctctctggta gaaaaatgaa gagggtcctg gtactactgc |
| 241 | ttgctgtggc atttggacat gctttagaga gaggccggga ttatgaaaag aataaagtct |
| 301 | gcaaggaatt ctcccatctg ggaaaggagg acttcacatc tctgtcacta gtcctgtaca |
| 361 | gtagaaaatt tcccagtggc acgtttgaac aggtcagcca acttgtgaag gaagttgtct |
| 421 | ccttgaccga agcctgctgt gcggaagggg ctgaccctga ctgctatgac accaggacct |
| 481 | cagcactgtc tgccaagtcc tgtgaaagta attctccatt ccccgttcac ccaggcactg |
| 541 | ctgagtgctg caccaaagag ggcctggaac gaaagctctg catggctgct ctgaaacacc |
| 601 | agccacagga attccctacc tacgtggaac ccacaaatga tgaaatctgt gaggcgttca |
| 661 | ggaaagatcc aaaggaatat gctaatcaat ttatgtggga atattccact aattacggac |
| 721 | aagctcctct gtcactttta gtcagttaca ccaagagtta tctttctatg gtagggtcct |
| 781 | gctgtacctc tgcaagccca actgtatgct ttttgaaaga gagactccag cttaaacatt |
| 841 | tatcacttct caccactctg tcaaatagag tctgctcaca atatgctgct tatggggaga |
| 901 | agaaatcaag gctcagcaat ctcataaagt tagcccaaaa agtgcctact gctgatctgg |
| 961 | aggatgtttt gccactagct gaagatatta ctaacatcct ctccaaatgc tgtgagtctg |
| 1021 | cctctgaaga ttgcatggcc aaagagctgc ctgaacacac agtaaaactc tgtgacaatt |
| 1081 | tatccacaaa gaattctaag tttgaagact gttgtcaaga aaaaacagcc atggacgttt |
| 1141 | ttgtgtgcac ttacttcatg ccagctgccc aactccccga gcttccagat gtagagttgc |
| 1201 | ccacaaacaa agatgtgtgt gatccaggaa acaccaaagt catggataag tatacatttg |
| 1261 | aactaagcag aaggactcat cttccggaag tattcctcag taaggtactt gagccaaccc |
| 1321 | taaaaagcct tggtgaatgc tgtgatgttg aagactcaac tacctgtttt aatgctaagg |
| 1381 | gccctctact aaagaaggaa ctatcttctt tcattgacaa gggacaagaa ctatgtgcag |
| 1441 | attattcaga aaatacattt actgagtaca agaaaaaact ggcagagcga ctaaaagcaa |
| 1501 | 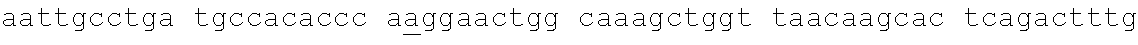 |
| 1561 | cctccaactg ctgttccata aactcacctc ctctttactg tgattcagag attgatgctg |
| 1621 | aattgaagaa tatcctgtag tcctgaagca tgtttattaa ctttgaccag agttggagcc |
| 1681 | acccagggga atgatctctg atgacctaac ctaagcaaaa ccactgagct tctgggaaga |
| 1741 | caactaggat actttctact ttttctagct acaatatctt catacaatga caagtatgat |
| 1801 | gatttgctat caaaataaat tgaaatataa tgcaaaccat aaaaaaaaaa aaaaaaaaaa |
| 1861 | aaaaaaaaaa aaaaaaaaaa aaaaaaaaaa aaaaa |

SEQ ID NO: 15 is the human amino acid sequence corresponding to the the vitamin D binding protein (GC), transcript variant 1 precursor (residues 1-474) having GenBank Accession No. NP_000574, where the italicized font corresponds to the presequence:

| | |
|---|---|
| 1 | MKRVLVLLLA VAFGHALERG RDYEKNKVCK EFSHLGKEDF TSLSLVLYSR KFPSGTFEQV |
| 61 | SQLVKEVVSL TEACCAEGAD PDCYDTRTSA LSAKSCESNS PFPVHPGTAE CCTKEGLERK |
| 121 | LCMAALKHQP QEFPTYVEPT NDEICEAFRK DPKEYANQFM WEYSTNYGQA PLSLLVSYTK |
| 181 | SYLSMVGSCC TSASPTVCFL KERLQLKHLS LLTTLSNRVC SQYAAYGEKK SRLSNLIKLA |
| 241 | QKVPTADLED VLPLAEDITN ILSKCCESAS EDCMAKELPE HTVKLCDNLS TKNSKFEDCC |
| 301 | QEKTAMDVFV CTYFMPAAQL PELPDVELPT NKDVCDPGNT KVMDKYTFEL SRRTHLPEVF |
| 361 | LSKVLEPTLK SLGECCDVED STTCFNAKGP LLKKELSSFI DKGQELCADY SENTFTEYKK |
| 421 | KLAERLKAKL PDATPTELAK LVNKHSDFAS NCCSINSPPL YCDSEIDAEL KNIL |

SEQ ID NO: 16 is the human nucleic acid sequence corresponding to the vitamin D binding protein (GC), transcript variant 1 precursor (bps 1-2024) having GenBank Accession No. NM_000583. The underlined ATG corresponds to the start codon, and the italicized font in the coding region corresponds to the presequence:

| | |
|---|---|
| 1 | atattaagta aactttagtg aggaacagca gtggaaaata atctatatac cttggctctt |
| 61 | ttgcagtttg acaaagttaa tgattaaaat ctcctagatt ttccactaca gtatccccag |
| 121 | ggtgtctatt taccttgatt gatattattt tatctctttt gggccaaaga taacagcccc |
| 181 | ttgcttctgt gtttaataat aattctgtgt tgcttctgag attaataatt gattaattca |
| 241 | tagtcaggaa tctttgtaaa aaggaaacca attacttttg gctaccactt ttacatggtc |
| 301 | acctacagga gagaggaggt gctgcaagac tctctggtag aaaaatgaag agggtcctgg |
| 361 | tactactgct tgctgtggca tttggacatg ctttagagag aggccgggat tatgaaaaga |
| 421 | ataaagtctg caaggaattc tcccatctgg gaaaggagga cttcacatct ctgtcactag |
| 481 | tcctgtacag tagaaaattt cccagtggca cgtttgaaca ggtcagccaa cttgtgaagg |
| 541 | aagttgtctc cttgaccgaa gcctgctgtg cggaaggggc tgaccctgac tgctatgaca |
| 601 | ccaggacctc agcactgtct gccaagtcct gtgaaagtaa ttctccattc cccgttcacc |
| 661 | caggcactgc tgagtgctgc accaaagagg gcctggaacg aaagctctgc atggctgctc |
| 721 | tgaaacacca gccacaggaa ttccctacct acgtggaacc cacaaatgat gaaatctgtg |
| 781 | aggcgttcag gaaagatcca aaggaatatg ctaatcaatt tatgtgggaa tattccacta |
| 841 | attacggaca agctcctctg tcacttttag tcagttacac caagagttat ctttctatgg |
| 901 | tagggtcctg ctgtacctct gcaagcccaa ctgtatgctt tttgaaagag agactccagc |
| 961 | ttaaacattt atcacttctc accactctgt caaatagagt ctgctcacaa tatgctgctt |
| 1021 | atggggagaa gaaatcaagg ctcagcaatc tcataaagtt agcccaaaaa gtgcctactg |
| 1081 | ctgatctgga ggatgttttg ccactagctg aagatattac taacatcctc tccaaatgct |
| 1141 | gtgagtctgc ctctgaagat tgcatggcca aagagctgcc tgaacacaca gtaaaactct |
| 1201 | gtgacaattt atccacaaag aattctaagt ttgaagactg ttgtcaagaa aaaacagcca |
| 1261 | tggacgtttt tgtgtgcact tacttcatgc cagctgccca actccccgag cttccagatg |
| 1321 | tagagttgcc cacaaacaaa gatgtgtgtg atccaggaaa caccaaagtc atggataagt |
| 1381 | atacatttga actaagcaga aggactcatc ttccggaagt attcctcagt aaggtacttg |
| 1441 | agccaaccct aaaaagcctt ggtgaatgct gtgatgttga agactcaact acctgtttta |
| 1501 | atgctaaggg ccctctacta aagaaggaac tatcttcttt cattgacaag ggacaagaac |
| 1561 | tatgtgcaga ttattcagaa aatacattta ctgagtacaa gaaaaaactg gcagagcgac |
| 1621 | taaaagcaaa attgcctgat gccacaccca cggaactggc aaagctggtt aacaagcact |
| 1681 | cagactttgc ctccaactgc tgttccataa actcacctcc tctttactgt gattcagaga |
| 1741 | ttgatgctga attgaagaat atcctgtagt cctgaagcat gtttattaac tttgaccaga |
| 1801 | gttggagcca cccaggggaa tgatctctga tgacctaacc taagcaaaac cactgagctt |
| 1861 | ctgggaagac aactaggata ctttctactt tttctagcta caatatcttc atacaatgac |
| 1921 | aagtatgatg atttgctatc aaaataaatt gaaatataat gcaaaccata aaaaaaaaaa |
| 1981 | aaaaaaaaaa aaaaaaaaaa aaaaaaaaaa aaaaaaaaaa aaaa |

SEQ ID NO: 17 is the human amino acid sequence corresponding to the the vitamin D binding protein (GC), transcript variant 3 precursor (residues 1-493) having GenBank Accession No. NP_001191236, where the italicized font corresponds to the presequence:

| | |
|---|---|
| 1 | MLWSWSEERG GAARLSGRKM KRVLVLLLAV AFGHALERGR DYEKNKVCKE FSHLGKEDFT |
| 61 | SLSLVLYSRK FPSGTFEQVS QLVKEVVSLT EACCAEGADP DCYDTRTSAL SAKSCESNSP |
| 121 | FPVHPGTAEC CTKEGLERKL CMAALKHQPQ EFPTYVEPTN DEICEAFRKD PKEYANQFMW |
| 181 | EYSTNYGQAP LSLLVSYTKS YLSMVGSCCT SASPTVCFLK ERLQLKHLSL LTTLSNRVCS |
| 241 | QYAAYGEKKS RLSNLIKLAQ KVPTADLEDV LPLAEDITNI LSKCCESASE DCMAKELPEH |
| 301 | TVKLCDNLST KNSKFEDCCQ EKTAMDVFVC TYFMPAAQLP ELPDVELPTN KDVCDPGNTK |
| 361 | VMDKYTFELS RRTHLPEVFL SKVLEPTLKS LGECCDVEDS TTCFNAKGPL LKKELSSFID |
| 421 | KGQELCADYS ENTFTEYKKK LAERLKAKLP DATPTELAKL VNKHSDFASN CCSINSPPLY |
| 481 | CDSEIDAELK NIL |

SEQ ID NO: 18 is the human nucleic acid sequence corresponding to the vitamin D binding protein (GC), transcript variant 3 precursor (bps 1-1832) having GenBank Accession No. NM_001204307. The underlined ATG corresponds to the start codon, and the italicized font in the coding region corresponds to the presequence:

| | |
|---|---|
| 1 | actgtttttt caaagagatt gcttactcca ccaggagaat gatatctacc tattctacca |
| 61 | ctggtgatga tctggactag tccaactggt aggaaatgct gtggtcttgg agtgaggaga |
| 121 | gaggaggtgc tgcaagactc tctggtagaa aaatgaagag ggtcctggta ctactgcttg |
| 181 | ctgtggcatt tggacatgct ttagagagag gccgggatta tgaaaagaat aaagtctgca |
| 241 | aggaattctc ccatctggga aaggaggact tcacatctct gtcactagtc ctgtacagta |
| 301 | gaaaatttcc cagtggcacg tttgaacagg tcagccaact tgtgaaggaa gttgtctcct |
| 361 | tgaccgaagc ctgctgtgcg gaaggggctg accctgactg ctatgacacc aggacctcag |
| 421 | cactgtctgc caagtcctgt gaaagtaatt ctccattccc cgttcaccca ggcactgctg |
| 481 | agtgctgcac caaagagggc ctggaacgaa agctctgcat ggctgctctg aaacaccagc |
| 541 | cacaggaatt ccctacctac gtggaaccca caaatgatga aatctgtgag gcgttcagga |
| 601 | aagatccaaa ggaatatgct aatcaattta tgtgggaata ttccactaat tacggacaag |
| 661 | ctcctctgtc acttttagtc agttacacca agagttatct ttctatggta gggtcctgct |
| 721 | gtacctctgc aagcccaact gtatgctttt tgaaagagag actccagctt aaacatttat |
| 781 | cacttctcac cactctgtca aatagagtct gctcacaata tgctgcttat ggggagaaga |
| 841 | aatcaaggct cagcaatctc ataaagttag cccaaaaagt gcctactgct gatctggagg |
| 901 | atgttttgcc actagctgaa gatattacta acatcctctc caaatgctgt gagtctgcct |
| 961 | ctgaagattg catggccaaa gagctgcctg aacacacagt aaaactctgt gacaatttat |
| 1021 | ccacaaagaa ttctaagttt gaagactgtt gtcaagaaaa aacagccatg gacgtttttg |
| 1081 | tgtgcactta cttcatgcca gctgcccaac tccccgagct tccagatgta gagttgccca |
| 1141 | caaacaaaga tgtgtgtgat ccaggaaaca ccaaagtcat ggataagtat acatttgaac |
| 1201 | taagcagaag gactcatctt ccggaagtat tcctcagtaa ggtacttgag ccaaccctaa |
| 1261 | aaagccttgg tgaatgctgt gatgttgaag actcaactac ctgttttaat gctaagggcc |
| 1321 | ctctactaaa gaaggaacta tcttctttca ttgacaaggg acaagaacta tgtgcagatt |
| 1381 | attcagaaaa tacatttact gagtacaaga aaaaactggc agagcgacta aaagcaaaat |
| 1441 | tgcctgatgc cacacccacg gaactggcaa agctggttaa caagcactca gactttgcct |
| 1501 | ccaactgctg ttccataaac tcacctcctc tttactgtga ttcagagatt gatgctgaat |
| 1561 | tgaagaatat cctgtagtcc tgaagcatgt ttattaactt tgaccagagt tggagccacc |
| 1621 | caggggaatg atctctgatg acctaaccta agcaaaacca ctgagcttct gggaagacaa |
| 1681 | ctaggatact ttctactttt tctagctaca atatcttcat acaatgacaa gtatgatgat |
| 1741 | ttgctatcaa aataaattga aatataatgc aaaccataaa aaaaaaaaaa aaaaaaaaaa |
| 1801 | aaaaaaaaaa aaaaaaaaaa aaaaaaaaaa aa |

SEQ ID NO: 14 is the human amino acid sequence corresponding to the the vitamin D binding protein (Gc globulin) (residues 1-458) having PDB accession no. 1J78_A:

| | |
|---|---|
| 1 | LERGRDYEKN KVCKEFSHLG KEDFTSLSLV LYSRKFPSGT FEQVSQLVKE VVSLTEACCA |
| 61 | EGADPDCYDT RTSALSAKSC ESNSPFPVHP GTAECCTKEG LERKLCMAAL KHQPQEFPTY |
| 121 | VEPTNDEICE AFRKDPKEYA NQFMWEYSTN YGQAPLSLLV SYTKSYLSMV GSCCTSASPT |
| 181 | VCFLKERLQL KHLSLLTTLS NRVCSQYAAY GEKKSRLSNL IKLAQKVPTA DLEDVLPLAE |
| 241 | DITNILSKCC ESASEDCMAK ELPEHTVKLC DNLSTKNSKF EDCCQEKTAM DVFVCTYFMP |
| 301 | AAQLPELPDV ELPTNKDVCD PGNTKVMDKY TFELSRRTHL PEVFLSKVLE PTLKSLGECC |
| 361 | DVEDSTTCFN AKGPLLKKEL SSFIDKGQEL CADYSENTFT EYKKKLAERL KAKLPDATP [symbol] |
| 421 | ELAKLVNKRS DFASNCCSIN SPPLYCDSEI DAELKNIL |

TABLE 1
Nucleotide and amino acid differences of the major Gc
variants. Underlined, bold text indicates the position
within the triplet of the nucleotide change in the coding
sequence (T→G at nucleotide position 1296, and C→A at
nucleotide position 1307 of the coding sequence).

| | Nucleotides | | Nucleotides | |
|---|---|---|---|---|
| | 1294-1296 | Corresponding | 1306-1308 | Corresponding |
| Gc | (T/G, position | amino acid | (C/A, position | amino acid |
| allele | 1296) | (residue 432) | 1307) | (residue 436) |
| Gc1f | GAT | Aspartic acid | ACG | Threonine (T) |
| | | (D) | | |
| Gc1s | GAG | Glutamic acid | ACG | Threonine (T) |
| | | (E) | | |
| Gc2 | GAT | Aspartic acid | AAG | Lysine (K) |
| | | (D) | | |

As used herein, a “Gc globulin molecule” means a nucleic acid which encodes a polypeptide that exhibits Gc globulin activity (e.g., vitamin D binding), or a polypeptide or peptidomimetic that exhibits Gc globulin activity. For example, a Gc globulin molecule can include the human Gc1F protein (e.g., having the amino acid sequence shown in SEQ ID NO: 1, 14, 15, or 17), or a variant thereof, such as a fragment thereof, that exhibits Gc globulin activity. For example, a Gc globulin molecule can include the human Gc1s protein (e.g., having the amino acid sequence shown in SEQ ID NO: 3), or a variant thereof, such as a fragment thereof, that exhibits Gc globulin activity. For example, a Gc globulin molecule can include the human Gc2 protein (e.g., having the amino acid sequence shown in SEQ ID NO: 5), or a variant thereof, such as a fragment thereof, that exhibits Gc globulin activity. The nucleic acid can be any type of nucleic acid, including genomic DNA, complementary DNA (cDNA), synthetic or semi-synthetic DNA, as well as any form of corresponding RNA. For example, a Gc globulin molecule can comprise a recombinant nucleic acid encoding human Gc protein, human Gc1s protein, or human Gc2 protein. In one embodiment, a Gc globulin molecule can comprise a non-naturally occurring nucleic acid created artificially (such as by assembling, cutting, ligating or amplifying sequences). A Gc globulin molecule can be double-stranded. A Gc globulin molecule can be single-stranded. The Gc globulin molecules of the invention can be obtained from various sources and can be produced according to various techniques known in the art. For example, a nucleic acid that is a Gc globulin molecule can be obtained by screening DNA libraries, or by amplification from a natural source. The Gc globulin molecules of the invention can be produced via recombinant DNA technology and such recombinant nucleic acids can be prepared by conventional techniques, including chemical synthesis, genetic engineering, enzymatic techniques, or a combination thereof. Non-limiting examples of a Gc globulin molecule that is a nucleic acid, is the nucleic acid comprising SEQ ID NO: 2, 4, 6, 8, 9, 10, 16 or 18. Another example of a Gc globulin molecule is a fragment of a nucleic acid comprising the sequence shown in SEQ ID NO: 2, 4, 6, 8, 9, 10, 16, or 18, wherein the fragment exhibits Gc globulin activity, e.g., vitamin D binding activity.

A Gc globulin molecule of this invention also encompasses variants of the human nucleic acid encoding the Gc1F, Gc1s, or Gc2 proteins that exhibit Gc globulin activity, or variants of the human Gc1F, Gc1s, or Gc2 proteins that exhibit Gc globulin activity. A Gc globulin molecule of this invention also includes a fragment of the human Gc1F, Gc1s, or Gc2 nucleic acid which encodes a polypeptide that exhibits vitamin D binding activity. A Gc globulin molecule of this invention encompasses a fragment of the human Gc1F, Gc1s, or Gc2 protein that exhibits Gc globulin activity, e.g., vitamin D binding activity.

The variants can comprise, for instance, naturally-occurring variants due to allelic variations between individuals (e.g., polymorphisms), mutated alleles related to autism or ASD, or alternative splicing forms. In one embodiment, a Gc globulin molecule is a nucleic acid variant of the nucleic acid having the sequence shown in SEQ ID NO: 2, wherein the variant has a nucleotide sequence identity to SEQ ID NO: 2 of at least about 65%, at least about 75%, at least about 85%, at least about 90%, at least about 91%, at least about 92%, at least about 93%, at least about 94%, at least about 95%, at least about 96%, at least about 97%, at least about 98%, or at least about 99% with SEQ ID NO: 2. In another embodiment, a Gc globulin molecule is a nucleic acid variant of the nucleic acid having the sequence shown in SEQ ID NO: 4, wherein the variant has a nucleotide sequence identity to SEQ ID NO: 4 of at least about 65%, at least about 75%, at least about 85%, at least about 90%, at least about 91%, at least about 92%, at least about 93%, at least about 94%, at least about 95%, at least about 96%, at least about 97%, at least about 98%, or at least about 99% with SEQ ID NO: 4. In one embodiment, a Gc globulin molecule is a nucleic acid variant of the nucleic acid having the sequence shown in SEQ ID NO: 6, wherein the variant has a nucleotide sequence identity to SEQ ID NO: 6 of at least about 65%, at least about 75%, at least about 85%, at least about 90%, at least about 91%, at least about 92%, at least about 93%, at least about 94%, at least about 95%, at least about 96%, at least about 97%, at least about 98%, or at least about 99% with SEQ ID NO: 6.

In one embodiment, a Gc globulin molecule encompasses any portion of at least about 8 consecutive nucleotides of SEQ ID NO: 2, 4, 6, 16, or 18. In one embodiment, the fragment can comprise at least about 15 nucleotides, at least about 20 nucleotides, or at least about 30 nucleotides of SEQ ID NO: 2, 4, 6, 16, or 18. Fragments include all possible nucleotide lengths between about 8 and 100 nucleotides, for example, lengths between about 15 and 100, or between about 20 and 100.

The invention further provides for nucleic acids that are complementary to a nucleic acid encoding Gc1F, Gc1s, or Gc2 proteins. Such complementary nucleic acids can comprise nucleic acid sequences, which hybridize to a nucleic acid sequence encoding a Gc1F, Gc1s, or Gc2 protein under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

In one embodiment, a Gc globulin molecule comprises a protein or polypeptide encoded by a Gc globulin nucleic acid sequence, such as the sequence shown in SEQ ID NO: 1, 3, 5, 7, or 9. In another embodiment, the polypeptide can be modified, such as by glycosylations and/or acetylations and/or chemical reaction or coupling, and can contain one or several non-natural or synthetic amino acids. An example of a Gc globulin molecule is the polypeptide having the amino acid sequence shown in SEQ ID NO: 1, 3, 5, 11, 12, 13, 14, 15, or 17.

In another embodiment, a Gc globulin molecule can be a fragment of a Gc globulin protein, such as Gc1F, Gc1s, or Gc2. For example, the Gc globulin molecule can encompass any portion of at least about 8 consecutive amino acids of SEQ ID NO: 1, 3, or 5. The fragment can comprise at least about 10 amino acids, a least about 20 amino acids, at least about 30 amino acids, at least about 40 amino acids, a least about 50 amino acids, at least about 60 amino acids, or at least about 75 amino acids of SEQ ID NO: 1, 3, or 5. Fragments include all possible amino acid lengths between about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100 amino acids, between about 70 and 100 amino acids, between about 75 and 100 amino acids, or between about 80 and 100 amino acids.

In certain embodiments, the Gc globulin molecule of the invention includes variants of the human Gc1F, Gc1s, or Gc2 protein (comprising the amino acid sequence shown in SEQ ID NO: 1, 3, or 5, respectively). Such variants can include those having at least from about 46% to about 50% identity to SEQ ID NO: 1, 3, or 5, or having at least from about 50.1% to about 55% identity to SEQ ID NO: 1, 3, or 5, or having at least from about 55.1% to about 60% identity to SEQ ID NO: 1, 3, or 5, or having from at least about 60.1% to about 65% identity to SEQ ID NO: 1, 3, or 5, or having from about 65.1% to about 70% identity to SEQ ID NO: 1, 3, or 5, or having at least from about 70.1% to about 75% identity to SEQ ID NO: 1, 3, or 5, or having at least from about 75.1% to about 80% identity to SEQ ID NO: 1, 3, or 5, or having at least from about 80.1% to about 85% identity to SEQ ID NO: 1, 3, or 5, or having at least from about 85.1% to about 90% identity to SEQ ID NO: 1, 3, or 5, or having at least from about 90.1% to about 95% identity to SEQ ID NO: 1, 3, or 5, or having at least from about 95.1% to about 97% identity to SEQ ID NO: 1, 3, or 5, or having at least from about 97.1% to about 99% identity to SEQ ID NO: 1, 3, or 5.

In another embodiment, the Gc globulin molecule of the invention encompasses a peptidomimetic which exhibits Gc globulin activity (e.g., vitamin D binding activity). A peptidomimetic is a small protein-like chain designed to mimic a peptide that can arise from modification of an existing peptide in order to protect that molecule from enzyme degradation and increase its stability, and/or alter the molecule's properties (e.g., modifications that change the molecule's stability or biological activity). These modifications involve changes to the peptide that cannot occur naturally (such as altered backbones and the incorporation of non-natural amino acids). Drug-like compounds can be developed from existing peptides. A peptidomimetic can be a peptide, partial peptide, or non-peptide molecule that mimics the tertiary binding structure or activity of a selected native peptide or protein functional domain (e.g., binding motif or active site). These peptide mimetic s include recombinantly or chemically modified peptides.

In one embodiment, a Gc globulin molecule comprising SEQ ID NO: 1, 3, or 5, variants of each, or fragments thereof, can be modified to produce peptide mimetics by replacement of one or more naturally occurring side chains of the 20 genetically encoded amino acids (or D amino acids) with other side chains. This can occur, for instance, with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, ifuryl, imidazolidinyl imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics can also have amino acid residues that have been chemically modified by phosphorylation, sulfonation, biotinylation, or the addition or removal of other moieties. For example, peptidomimetics can be designed and directed to amino acid sequences encoded by a Gc globulin molecule comprising SEQ ID NO: 1, 3, 5, 14, 15, or 17.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24, 243-252, 1989). Certain peptidomimetic compounds are based upon the amino acid sequence of the peptides of the invention. Peptidomimetic compounds can be synthetic compounds having a three-dimensional structure (i.e. a peptide motif) based upon the three-dimensional structure of a selected peptide. The peptide motif provides the peptidomimetic compound with the desired biological activity, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life. Peptidomimetic design strategies are readily available in the art (see, e.g., Ripka & Rich (1998) Curr. Op. Chem. Biol. 2:441-452; Hruby et al. (1997) Curr. Op. Chem. Biol. 1:114-119; Hruby & Balse, (2000) Curr. Med. Chem. 9:945-970).

Gc Globulin Modulating Compounds

As used herein, a “Gc Globulin modulating compound” refers to a compound that interacts with a Gc globulin polypeptide molecule (e.g., Gc1F, Gc1s, or Gc2) and modulates its vitamin D binding activity and/or its expression. The compound can either increase a Gc globulin's activity or expression. Conversely, the compound can decrease Gc globulin's activity or expression. The compound can be a Gc globulin agonist or a Gc globulin antagonist. Some non-limiting examples of Gc globulin modulating compounds include peptides (such as Gc globulin peptide fragments, or antibodies or fragments thereof), small molecules, and nucleic acids (such as Gc globulin siRNA or antisense RNA specific for a Gc globulin nucleic acid). Agonists of a Gc globulin molecule can be molecules which, when bound to Gc globulin, increase or prolong the activity of a Gc globulin molecule. Agonists of Gc globulin include, but are not limited to, proteins, nucleic acids, small molecules, or any other molecule which activates Gc globulin. Antagonists of a Gc globulin molecule can be molecules which, when bound to Gc globulin or a variant thereof, decrease the amount or the duration of the activity of a Gc globulin molecule. Antagonists include proteins, nucleic acids, antibodies, small molecules, or any other molecule which decrease the activity of Gc globulin.

The term “modulate,” as it appears herein, refers to a change in the activity or expression of a Gc globulin molecule. For example, modulation can cause an increase or a decrease in protein activity, binding characteristics, or any other biological, functional, or immunological properties of a Gc globulin molecule.

In one embodiment, a Gc globulin modulating compound can be a peptide fragment of a Gc globulin protein that binds to the protein. For example, the Gc globulin molecule can encompass any portion of at least about 8 consecutive amino acids of SEQ ID NO: 1, 3, 5, 11, 12, 13, 14, 15, or 17. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 1, 3, 5, 14, 15, or 17. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The Gc globulin peptide fragments can be isolated from a natural source, genetically engineered, or chemically prepared. These methods are well known in the art.

A Gc globulin modulating compound can also be a protein, such as an antibody (monoclonal, polyclonal, humanized, chimeric, or fully human), or a binding fragment thereof, directed against a Gc globulin (e.g, Gc1F, Gc1s, or Gc2 having SEQ ID NO: 1, 3, or 5, respectively). In one embodiment, the Gc globulin comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (see Roland E. Kontermann and Stefan Dübel (editors), Antibody Engineering, Vol. I & II, (2010) 2^{nd} ed., Springer; Antony S. Dimitrov (editor), Therapeutic Antibodies: Methods and Protocols (Methods in Molecular Biology), (2009), Humana Press; Benny Lo (editor) Antibody Engineering: Methods and Protocols (Methods in Molecular Biology), (2004) Humana Press, each of which are hereby incorporated by reference in their entirities).

Inhibition of RNA encoding a Gc globulin (e.g., Gc1F, Gc1s, or Gc2) can effectively modulate the expression of the Gc globulin gene from which the RNA is transcribed. Inhibitors are selected from the group comprising: siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a Gc globulin polypeptide (e.g., comprising SEQ ID NO: 2, 4, 6, 8, 9, or 10) can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like.

An siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McMnaus and Sharp (2002) Nat Rev Genetics, 3:737-47, and Sen and Blau (2006) FASEB J., 20:1293-99, the entire disclosures of which are herein incorporated by reference.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. No. 7,294,504 and U.S. Pat. No. 7,422,896, the entire disclosures of which are hereby incorporated by reference in their entireties). Exemplary methods for producing and testing dsRNA or siRNA molecules are described in U.S. Patent Application Publication No. 2002/0173478 to Gewirtz, U.S. Pat. No. 8,071,559 to Hannon et al., U.S. Pat. No. 7,674,895 to Reich et al., and in U.S. Pat. No. 7,148,342 to Tolentino et al., the entire disclosures of which each are hereby incorporated by reference.

In one embodiment, an siRNA directed to human Gc globulin comprises a nucleic acid directed to any one of SEQ ID NOS: 2, 4, 6, 8, 9, or 10.

A Gc globulin modulating compound can also be a small molecule that binds to Gc globulin and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate Gc globulin can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries known in the art (for example, see Werner et al., (2006) Brief Funct. Genomic Proteomic 5(1):32-6).

Diagnosis

The invention provides diagnosis methods based on monitoring a gene encoding a Gc globulin molecule (such as Gc1F, Gc1s, or Gc2). As used herein, the term “diagnosis” includes the detection, typing, monitoring, dosing, comparison, at various stages, including early, pre-symptomatic stages, and late stages, in adults, children, and unborn human children. Diagnosis can include the assessment of a predisposition or risk of development, the prognosis, or the characterization of a subject to define most appropriate treatment (pharmacogenetics).

The invention provides diagnostic methods to determine whether an individual is at risk of developing autism or an autism spectrum disorder (ASD), or suffers from autism or an ASD, wherein the disease reflects an alteration in the expression of a gene encoding a Gc globulin molecule (such as Gc1F, Gc1s, or Gc2). Subjects diagnosed with autism, as well as ASD, can display some core symptoms in the areas of a) social interactions and relationships, b) verbal and non-verbal communication, and c) physical activity, play, physical behavior. For example, symptoms related to social interactions and relationships can include but are not limited to the inability to establish friendships with children the same age, lack of empathy, and the inability to develop nonverbal communicative skills (for example, eye-to-eye gazing, facial expressions, and body posture). For example, symptoms related to verbal and nonverbal communication comprises delay in learning to talk, inability to learn to talk, failure to initiate or maintain a conversation, failure to interpret or understand implied meaning of words, and repetitive use of language. For example, symptoms related to physical activity, play, physical behavior include, but are not limited to unusual focus on pieces or parts of an object, such as a toy, a preoccupation with certain topics, a need for routines and rituals, and stereotyped behaviors (for example, body rocking and hand flapping).

In one embodiment, a method of detecting the presence of or a predisposition to autism or an autism spectrum disorder in a subject is provided. The subject can be a human (e.g., a pregnant female) or a child thereof. In one embodiment, the subject is of non-caucasian origin. The subject can also be a human embryo, a human fetus, or an unborn human child. In one embodiment, the method can comprise detecting in a sample whether or not there is a nucleic acid sequence encoding a Gc globulin protein having SEQ ID NO: 2, 4, 6, 8, 9, or 10. In some instances, the presence of a certain allele, e.g., the ancestral Gc1F allele, can also be detected. In one embodiment, the method can comprise detecting in a sample from the subject the presence of an alteration in the expression of a gene of a Gc globulin molecule (such as Gc1F, Gc1s, or Gc2). In one embodiment, the detecting comprises detecting whether there is an alteration in the gene locus encoding a Gc globulin molecule (e.g., Gc1F, Gc1s, or Gc2) in a sample obtained from a subject. In another embodiment, the detecting comprises detecting whether expression of a Gc globulin molecule (e.g., Gc1F, Gc1s, or Gc2) is increased. In a further embodiment, the detecting comprises detecting whether expression of a Gc globulin molecule (such as Gc1s, or Gc2) is reduced. In some embodiments, the detecting comprises detecting in the sample whether there is an increase in an mRNA encoding a Gc globulin molecule (e.g., Gc1F, Gc1s, or Gc2), or an increase in the Gc globulin protein, or a combination thereof. In some embodiments, the detecting comprises detecting in the sample whether there is a reduction in an mRNA encoding a Gc globulin molecule (e.g., Gc1s or Gc2), or a reduction in the Gc globulin protein, or a combination thereof. The presence of such an alteration is indicative of the presence or predisposition to autism or an autism spectrum disorder.

In another embodiment, a method of detecting the presence of or a predisposition to vitamin D deficiency-related neurodevelopmental disorder in a subject is provided. Non-limiting examples of vitamin D deficiency-related neurodevelopemental disorders include an increase in volume of lateral ventricles, a decrease in brain cortex thickness, a decrease in neurite outgrowth, an increase in dentate gyrus mitotic cells, an increase in basal ganglia mitotic cells, an increase in hypothalamus mitotic cells, a decrease in calcium uptake, a decrease in dentate gyrus apoptotic cells, a decrease in basal ganglia apoptotic cells, a decrease in hypothalamus apoptotic cells, an increase in subventricular zone (SVZ) neurospheres, a decrease in levels of nerve growth factor (NGF), a decrease in levels of Glial cell-derived neurotrophic factor (GDNF), a decrease in levels of nurr1 transcription factor, a decrease in levels of p75 neurotrophin receptor (p75NTR), a decrease in levels of catechol-O-methyltransferase (COMT), a decrease in levels of neurotrophin-3 (NT-3), and a decrease in levels of neurotrophin-4 (NT-4). See Harms et al., Best Pract Res Clin Endocrinol Metab. 2011; 25:657-669; Eyles D. et al., Semin Cell Dev Biol. 2011; 22:629-636; and Eyles D W, et al., Psychoneuroendocrinology. 2009; 34 Suppl 1:S247-S257, each of which are hereby incorporated by reference in their entireties.

The subject can be a human (e.g., a pregnant female) or a child thereof. In one embodiment, the subject is of non-caucasian origin. The subject can also be a human embryo, a human fetus, or an unborn human child. In one embodiment, the method can comprise detecting in a sample whether or not there is a nucleic acid sequence encoding a Gc globulin protein having SEQ ID NO: 2, 4, 6, 8, 9, or 10. In some instances, the presence of a certain allele, e.g., the ancestral Gc1F allele, can also be detected. In one embodiment, the method can comprise detecting in a sample from the subject the presence of an alteration in the expression of a gene of a Gc globulin molecule (such as Gc1F, Gc1s, or Gc2). In one embodiment, the detecting comprises detecting whether there is an alteration in the gene locus encoding a Gc globulin molecule (e.g., Gc1F, Gc1s, or Gc2) in a sample obtained from a subject. In another embodiment, the detecting comprises detecting whether expression of a Gc globulin molecule (e.g., Gc1F, Gc1s, or Gc2) is increased. In a further embodiment, the detecting comprises detecting whether expression of a Gc globulin molecule (such as Gc1s, or Gc2) is reduced. In some embodiments, the detecting comprises detecting in the sample whether there is an increase in an mRNA encoding a Gc globulin molecule (e.g., Gc1F, Gc1s, or Gc2), or an increase in the Gc globulin protein, or a combination thereof. In some embodiments, the detecting comprises detecting in the sample whether there is a reduction in an mRNA encoding a Gc globulin molecule (e.g., Gc1s or Gc2), or a reduction in the Gc globulin protein, or a combination thereof. The presence of such an alteration is indicative of the presence or predisposition to a vitamin D deficiency-related neurodevelopemental disorder.

In a further embodiment, a method of detecting the presence of or a predisposition to vitamin D deficiency-related immune deficit in a subject is provided. Non-limiting examples of vitamin D deficiency-related immune deficits include type I diabetes mellitus, multiple sclerosis, rheumatoid arthritis, systemic lupus erythematosus, inflammatory bowel disease, asthma, a bacterial infection, a viral infection. See Di Rosa M, et al., Immunology. 2011; 134:123-139; and Van Belle et al., Best Pract Res Clin Endocrinol Metab. 2011; 25:617-632, each of which are hereby incorporated by reference in their entireties. In one embodiment, the bacterial infection comprises a Mycobacterium tuberculosis infection, a Bordatella bronchi septica infection, a Pseudomonas aeruginosa infection, a Listeria monocytogenes infection, a Group B Streptococcus infection, or a combination thereof. In another embodiment, the viral infection comprises an influenza infection, a hepatitis C infection, an HIV-1 infection, or a combination thereof. The subject can be a human (e.g., a pregnant female) or a child thereof. In one embodiment, the subject is of non-caucasian origin. The subject can also be a human embryo, a human fetus, or an unborn human child. In one embodiment, the method can comprise detecting in a sample whether or not there is a nucleic acid sequence encoding a Gc globulin protein having SEQ ID NO: 2, 4, 6, 8, 9, or 10. In some instances, the presence of a certain allele, e.g., the ancestral Gc1F allele, can also be detected. In one embodiment, the method can comprise detecting in a sample from the subject the presence of an alteration in the expression of a gene of a Gc globulin molecule (such as Gc1F, Gc1s, or Gc2). In one embodiment, the detecting comprises detecting whether there is an alteration in the gene locus encoding a Gc globulin molecule (e.g., Gc1F, Gc1s, or Gc2) in a sample obtained from a subject. In another embodiment, the detecting comprises detecting whether expression of a Gc globulin molecule (e.g., Gc1F, Gc1s, or Gc2) is increased. In a further embodiment, the detecting comprises detecting whether expression of a Gc globulin molecule (such as Gc1s, or Gc2) is reduced. In some embodiments, the detecting comprises detecting in the sample whether there is an increase in an mRNA encoding a Gc globulin molecule (e.g., Gc1F, Gc1s, or Gc2), or an increase in the Gc globulin protein, or a combination thereof. In some embodiments, the detecting comprises detecting in the sample whether there is a reduction in an mRNA encoding a Gc globulin molecule (e.g., Gc1s or Gc2), or a reduction in the Gc globulin protein, or a combination thereof. The presence of such an alteration is indicative of the presence or predisposition to a vitamin D deficiency-related immune deficit.

The presence of an alteration in a gene encoding a Gc globulin molecule in the sample can be detected through the genotyping of a sample, for example via gene sequencing, selective hybridization, amplification, gene expression analysis, or a combination thereof. In one embodiment, the sample can comprise cord blood, blood, plasma, serum, cerebrospinal fluid, sputum, lacrimal secretions, semen, vaginal secretions, fetal tissue, skin tissue, muscle tissue, amniotic fluid, or a combination thereof.

The alteration can be determined at the DNA, RNA, or polypeptide level of the Gc globulin. The detection can also be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (e.g., see Jones et al., (2000) Hum Genet., 106(6):663-8), or a combination thereof. In some embodiments, the detection is performed by sequencing all or part of a Gc globulin gene or by selective hybridization or amplification of all or part of a Gc globulin gene. A Gc globulin gene specific amplification can be carried out before the alteration identification step.

An alteration in a Gc globulin gene locus (e.g., encoding Gc1F, Gc1s, or Gc2) can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs, although larger deletions can occur as well. Rearrangement includes inversion of sequences. The Gc globulin gene locus alteration can result in amino acid substitutions, RNA splicing or processing, product instability, the creation of stop codons, frame-shift mutations, and/or truncated polypeptide production. The alteration can result in the production of a Gc globulin polypeptide with altered function, stability, targeting or structure. The alteration can cause a reduction in protein expression, while in other instances the alteration can cause an increase in protein expression. In one embodiment, the alteration in a Gc globulin gene locus can comprise a point mutation, a deletion, or an insertion in the Gc globulin gene or corresponding expression product. In one embodiment, the alteration can be a deletion or partial deletion of a Gc globulin gene. The alteration can be determined at the level of the DNA, RNA, or polypeptide of a Gc globulin. In one embodiment, the method comprises detecting whether levels of serum actin-complexed Gc globulin protein are increased; levels of serum actin-free Gc globulin protein are increased; the glycosylation of actin-complexed Gc globulin is increased; the glycosylation of actin-free Gc globulin is increased; the sialylation of actin-complexed Gc globulin is increased; the sialylation of actin-free Gc globulin is increased; or a combination thereof.

In another embodiment, the method can comprise detecting the presence of an altered RNA expression of a Gc globulin. Altered RNA expression includes the presence of an altered RNA sequence, the presence of an altered RNA splicing or processing, or the presence of an altered quantity of RNA. These can be detected by various techniques known in the art, including by sequencing all or part of the RNA of a Gc globulin, or by selective hybridization or selective amplification of all or part of the RNA. In a further embodiment, the method can comprise detecting the presence of altered polypeptide expression of a Gc globulin. Altered polypeptide expression includes the presence of an altered polypeptide sequence, the presence of an altered quantity of Gc globulin polypeptide, or the presence of an altered tissue distribution. These can be detected by various techniques known in the art, including by sequencing and/or binding to specific ligands (such as antibodies, e.g., directed to a Gc globulin). In one embodiment, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof.

Various techniques known in the art can be used to detect or quantify altered gene expression, RNA expression, or sequence. These techniques include, but are not limited to, hybridization, sequencing, amplification, and/or binding to specific ligands (such as antibodies).

Hybridization detection methods are based on the formation of specific hybrids between complementary nucleic acid sequences that serve to detect nucleic acid sequence alteration(s). A detection technique involves the use of a nucleic acid probe specific for a wild type or altered gene or RNA, followed by the detection of the presence of a hybrid. The probe can be in suspension or immobilized on a substrate or support (for example, as in nucleic acid array or chips technologies). The probe can be labeled to facilitate detection of hybrids. In one embodiment, the probe according to the invention can comprise a nucleic acid directed to SEQ ID NOS: 2, 4, 6, 8, 9, or 10. For example, a sample from the subject can be contacted with a nucleic acid probe specific for a wild type Gc globulin gene or an altered Gc globulin gene, and the formation of a hybrid can be subsequently assessed. In one embodiment, the method comprises contacting simultaneously the sample with a set of probes that are specific for the wild type Gc globulin gene and for various gene alterations. Thus, it is possible to detect directly the presence of various forms of alterations in the Gc globulin gene (e.g., Gc1F, Gc1s, or Gc2) in the sample. Also, various samples from various subjects can be investigated in parallel.

For example, the following probes directed to Gc1s are available:

1. Bead microarray element (bead) probe for Homo sapiens variation rs7041. Reagent is available from Illumina. Probe: Pr008430515.1 http://www.ncbi.nlm.nih.gov/projects/genome/probe/reports/probereport.cgi?uid=8430515

2. Bead microarray element (bead) probe for Homo sapiens variation rs7041. Reagent is available from Illumina. Probe: Pr007807031.1 http://www.ncbi.nlm.nih.gov/projects/genome/probe/reports/probereport.cgi?uid=7807031

3. Bead microarray element (bead) probe for Homo sapiens variation rs7041. Reagent is available from Illumina. Probe: Pr007318863.1 http://www.ncbi.nlm.nih.gov/projects/genome/probe/reports/probereport.cgi?uid=7318863

4. Submitdb brokering default probe type (generic) rs7041-126_B_F_IFB1135765808:0 for variation rs7041. Probe: Pr011334781.2 http://www.ncbi.nlm.nih.gov/projects/genome/probe/reports/probereport.cgi?uid=11334781

5. Resequencing amplicon (RSA) probe RSA000472488 for Homo sapiens gene vitamin D-binding protein (GC). Developed for SNP discovery, validation succeeded. Probe: Pr001403066.1 http://www.ncbi.nlm.nih.gov/projects/genome/probe/reports/probereport.cgi?uid=1403066

6. Resequencing amplicon (RSA) probe RSA001009869 for Homo sapiens gene vitamin D-binding protein (GC). Probe: Pr001398777.1 http://www.ncbi.nlm.nih.gov/projects/genome/probe/reports/probereport.cgi?uid=1398777

7. Bead microarray element (bead) probe for Homo sapiens variation rs7041. Has been used in the HapMap project for genotyping. Reagent is available from Illumina. Probe: Pr435665.1 http://www.ncbi.nlm.nih.gov/projects/genome/probe/reports/probereport.cgi?uid=435665

For example, the following probes directed to Gc2 are available:

1. Sequence-specific oligonucleotide (SSO) probe for Homo sapiens variation rs4588. Has been used in the HapMap project for genotyping. Reagent is available from Perlegen. Probe: Pr002679946.1 http://www.ncbi.nlm.nih.gov/projects/genome/probe/reports/probereport.cgi?uid=2679946

2. Resequencing amplicon (RSA) probe RSA000472488 for Homo sapiens gene vitamin D-binding protein (GC). Developed for SNP discovery, validation succeeded. Probe: Pr001403066.1 http://www.ncbi.nlm.nih.gov/projects/genome/probe/reports/probereport.cgi?uid=1403066

3. Resequencing amplicon (RSA) probe RSA001009869 for Homo sapiens gene vitamin D-binding protein (GC). Probe: Pr001398777.1 http://www.ncbi.nlm.nih.gov/projects/genome/probe/reports/probereport.cgi?uid=1398777

4. Bead microarray element (bead) probe for Homo sapiens variation rs4588. Has been used in the HapMap project for genotyping. Reagent is available from Illumina. Probe: Pr470129.1 http://www.ncbi.nlm.nih.gov/projects/genome/probe/reports/probereport.cgi?uid=470129

According to the invention, a probe can be a polynucleotide sequence which is complementary to and specifically hybridizes with a, or a target portion of a, Gc globulin gene or RNA. These probes are suitable for detecting polynucleotide polymorphisms associated with alleles of a Gc globulin, which predispose to or are associated with autism or an autism spectrum disorder. Useful probes are those that are complementary to the Gc globulin gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a gene or RNA that carries an alteration.

The sequence of the probes can be derived from the sequences of the Gc globulin genes provided herein. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., Molecular Cloning: A Laboratory Manual (3^{rd} Ed.), Vols. 1-3, Cold Spring Harbor Laboratory, 1989; Current Protocols In Molecular Biology, Ausubel, ed. John Wiley & Sons, Inc., New York, 2001; Laboratory Techniques In Biochemistry And Molecular Biology: Hybridization With Nucleic Acid Probes, Part I. Theory and Nucleic Acid Preparation, Tijssen, ed. Elsevier, N.Y., 1993.

Sequencing can be carried out using techniques well known in the art, using automatic sequencers. The sequencing can be performed on the complete gene or on specific domains thereof, such as those known or suspected to carry deleterious mutations or other alterations.

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR based single-strand conformational polymorphism (SSCP). Amplification usually requires the use of specific nucleic acid primers, to initiate the reaction. For example, nucleic acid primers useful for amplifying sequences from the gene or locus of a Gc globulin (such as Gc1F, Gc1s, or Gc2) are able to specifically hybridize with a portion of the gene locus that flanks a target region of the locus, wherein the target region is altered in certain subjects having autism or an autism spectrum disorder. In one embodiment, amplification comprises using forward and reverse PCR primers directed to SEQ ID NOS: 2, 4, 6, 8, 9, or 10. Non-limiting amplification methods include, e.g., polymerase chain reaction, PCR (PCR Protocols, A Guide To Methods And Applications, ed. Innis, Academic Press, N.Y., 1990 and PCR Strategies, 1995, ed. Innis, Academic Press, Inc., N.Y.); ligase chain reaction (LCR) (Wu (1989) Genomics 4:560; Landegren (1988) Science 241:1077; Barringer (1990) Gene 89:117); transcription amplification (Kwoh (1989) PNAS 86:1173); and, self-sustained sequence replication (Guatelli (1990) PNAS 87:1874); Q Beta replicase amplification (Smith (1997) J. Clin. Microbiol. 35:1477-1491), automated Q-beta replicase amplification assay (Burg (1996) Mol. Cell. Probes 10:257-271) and other RNA polymerase mediated techniques (e.g., NASBA, Cangene, Mississauga, Ontario; see also Berger (1987) Methods Enzymol. 152:307-316; U.S. Pat. Nos. 4,683,195 and 4,683,202; and Sooknanan (1995) Biotechnology 13:563-564). All the references stated above are incorporated by reference in their entireties.

The invention provides for a nucleic acid primer, wherein the primer can be complementary to and hybridize specifically to a portion of a coding sequence (e.g., gene or RNA) of a Gc globulin (such as Gc1F, Gc1s, or Gc2) that is altered in certain subjects having autism or an autism spectrum disorder. Primers of the invention can thus be specific for altered sequences in a gene or RNA of a Gc globulin. By using such primers, the detection of an amplification product indicates the presence of an alteration in the gene or the absence of such gene. Examples of primers of this invention can be single-stranded nucleic acid molecules of about 5 to 60 nucleotides in length, or about 8 to about 25 nucleotides in length. The sequence can be derived directly from the sequence of the Gc globulin (e.g., Gc1F, Gc1s, or Gc2). Perfect complementarity is useful to ensure high specificity. However, certain mismatch can occur. In one embodiment, the primer can be an isolated nucleic acid directed to SEQ ID NOS: 2, 4, 6, 8, 9, or 10. For example, a nucleic acid primer or a pair of nucleic acid primers as described above can be used in a method for detecting the presence of or a predisposition to autism or an autism spectrum disorder in a subject. In one embodiment, the detecting comprises using hybridization, amplification, or sequencing techniques to distinguish SEQ ID NOS: 2, 4, 6, 8, 9, and/or 10.

Other suitable methods used to detect or quantify altered gene expression, RNA expression, or sequence include, but are not limited to, allele-specific oligonucleotide (ASO), oligonucleotide ligation, allele-specific amplification, Southern blot (for DNAs), Northern blot (for RNAs), single-stranded conformation analysis (SSCA), pulsed-field gel electrophoresis (PFGE), isoelectric focusing, fluorescent in situ hybridization (FISH), gel migration, clamped denaturing gel electrophoresis, denaturing HPLC, melting curve analysis, heteroduplex analysis, RNase protection, chemical or enzymatic mismatch cleavage, ELISA, radio-immunoassays (RIA) and immuno-enzymatic assays (IEMA). In one embodiment, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof.

Some of these approaches (such as SSCA and constant gradient gel electrophoresis (CGGE)) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody. In one embodiment, the antibody is directed to a Gc globulin comprising SEQ ID NO: 1, 3, 5, 11, 12, 13, 14, 15, or 17.

As indicated herein, alteration in a Gc globulin gene locus or in Gc globulin expression can also be detected by screening for alteration(s) in corresponding polypeptide sequence or expression levels. Different types of ligands can be used, such as specific antibodies. In one embodiment, the sample is contacted with an antibody specific for a Gc globulin or Gc globulin modulating polypeptide and the formation of an immune complex is subsequently determined. Various methods for detecting an immune complex can be used, such as ELISA, radioimmunoassays (RIA) and immuno-enzymatic assays (IEMA). In one embodiment, levels are measured by ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof.

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a Gc globulin polypeptide can be an antibody that selectively binds a Gc globulin polypeptide, for example, an antibody raised against a Gc globulin polypeptide or an epitope-containing fragment thereof. Although non-specific binding towards other antigens can occur, binding to the target polypeptide occurs with a higher affinity and can be reliably discriminated from non-specific binding. In one embodiment, the method comprises contacting a sample from the subject with an antibody specific for a wild type or an altered form of a Gc globulin polypeptide, and determining the presence of an immune complex. Optionally, the sample can be contacted to a support coated with antibody specific for the wild type or altered form of a Gc globulin polypeptide. In one embodiment, the sample can be contacted simultaneously, or in parallel, or sequentially, with various antibodies specific for different forms of a Gc globulin polypeptide, such as a wild type and various altered forms thereof.

As indicated herein, alteration in a Gc globulin gene locus or in Gc globulin expression can also be detected by screening for alteration(s) in corresponding polypeptide sequence or expression levels, or by measuring the levels of peptides associated with the Gc variants. In some embodiments, the Gc variant type is determined. In some embodiments, the Gc variant type is quantitated. In some embodiments, Gc variant type or level is determined by measuring the levels of peptides associated with a Gc variant type. In some embodiments, the Gc variant type is determined using mass spectrometry. In some embodiments, the Gc variant type is determined using LC-MS/MS mass spectrometry. In some embodiments, the levels of peptides associated with a Gc variant type is determined using mass spectrometry. In some embodiments, the levels of peptides associated with a Gc variant type is determined using LC-MS/MS mass spectrometry. In some embodiments, the levels of peptides associated with a Gc variant type is determined using multiple reaction monitoring (MRM). In some embodiments, the levels of peptides associated with a Gc variant type in a sample are compared to peptide levels in a sample from a non-autistic subject. In some embodiments, the sample is a serum sample. In some embodiments, the serum sample depleted of abundant plasma proteins. Methods to deplete abundant plasma proteins are known to one of skill in the art and include, but are not limited to, affinity chromatography. In some embodiments, the serum sample is not depleted of abundant plasma proteins.

The invention also provides for the determination of vitamin D status. As indicated herein, vitamin D status can be determined by detecting the Gc variant type in addition to measuring vitamin D levels. Determination of Gc globulin variant type and measurement of levels of Gc globulin as described herein can be used for accurate assessment of vitamin D status. In some embodiments, measurements of vitamin D status includes, but is not limited to, determination of Gc globulin variant type, analysis of levels of Gc globulin as well as measurements of total and free vitamin D levels. In some embodiments, analysis of vitamin D levels (vitamin D3, 25-hydroxyvitamin D3 and/or 24,25-dihydroxyvitamin D3) can be performed by LC-MS/MS targeting vitamin D and its metabolites (Burild et al. 2014). In other embodiments, total vitamin D levels (vitamin D3, 25-hydroxyvitamin D3 and/or 24,25-dihydroxyvitamin D3) can be measured by immunoassays including ELISA using an antibody directed to vitamin D; western blot using an antibody directed to vitamin D; or bead-based immunoassays available commercially, such as from Luminex, or other related detection platforms.

In some embodiments, detection can be performed in samples from maternal samples during pregnancy, at birth, and at timepoints after birth, in samples from children at birth and at timepoints after birth, in samples from fathers prior to conception, at conception and at timepoints after birth. In some embodiments, determination of maternal and paternal Gc globulin variant type prior to conception or early in pregnancy can allow for early identification of pregnancies that may require closer and more accurate monitoring of vitamin D status.

Accordingly, in one aspect, the invention provides a method for detecting the presence of or a predisposition to autism or an autism spectrum disorder (ASD) in a human subject or a child of a human subject. In some embodiments, the method comprises: (a) obtaining a biological sample from a human subject; (b) detecting whether or not there is an alteration in the expression of a Gc globulin protein in the subject as compared to a non-autistic subject; and (c) measuring vitamin D levels in the subject. In other embodiments, the method comprises: (a) obtaining a biological sample from a human subject; (b) detecting whether or not there is a nucleic acid sequence encoding a Gc globulin protein having the sequence of SEQ ID NO: 2, 4, 6, 8, 9, or 10, or a combination thereof; and (c) measuring vitamin D levels in the subject. In some embodiments, the subject is a pregnant female. In other embodiments, the subject is a child of a human subject, while yet in further embodiments, the subject is a human embryo, a human fetus, or an unborn human child. In some embodiments, the method further comprises detecting whether there is an alteration in a gene locus that encodes the Gc globulin protein. In other embodiments, the detecting comprises using PCR and primers directed to SEQ ID NO: 2, 4, 6, 8, 9, or 10. In further embodiments, the detecting comprises using hybridization, amplification, or sequencing techniques to distinguish SEQ ID NOS: 2, 4, 6, 8, 9, and/or 10. In some embodiments, the detecting comprises detecting whether mRNA expression of the Gc globulin protein is increased. In further embodiments, the detecting comprises using a northern blot; real time PCR and primers directed to SEQ ID NO: 2, 4, 6, 8, 9, or 10; a ribonuclease protection assay; a hybridization, amplification, or sequencing technique to distinguish SEQ ID NOS: 2, 4, 6, 8, 9, and/or 10; or a combination thereof. In other embodiments, the detecting comprises detecting whether levels of serum actin-complexed Gc globulin protein are increased; levels of serum actin-free Gc globulin protein are increased; the glycosylation of actin-complexed Gc globulin is increased; the glycosylation of actin-free Gc globulin is increased; the sialylation of actin-complexed Gc globulin is increased; the sialylation of actin-free Gc globulin is increased; or a combination thereof. In some embodiments, Gc globulin levels are measured by ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof. In further embodiments, the Gc globulin protein comprises Gc1s, Gc2, Gc1f, or a combination thereof. In some embodiments, the sample comprises blood, plasma, serum, cerebrospinal fluid, sputum, lacrimal secretions, semen, vaginal secretions, fetal tissue, liver tissue, amniotic fluid, cord blood, or a combination thereof. In some embodiments, measurements of vitamin D levels comprises measuring total and free vitamin D levels. In some embodiments, analysis of vitamin D levels (vitamin D3, 25-hydroxyvitamin D3 and/or 24,25-dihydroxyvitamin D3) can be performed by LC-MS/MS targeting vitamin D and its metabolites (Burild et al. 2014). In other embodiments, total vitamin D levels (vitamin D3, 25-hydroxyvitamin D3 and/or 24,25-dihydroxyvitamin D3) can be measured by immunoassays including ELISA using an antibody directed to vitamin D; western blot using an antibody directed to vitamin D; or bead-based immunoassays available commercially, such as from Luminex, or other related detection platforms. In some embodiments, the method further comprises assessing whether or not the course of autism or ASD will be severe. For example, severity can be assessed clinically by scores on the Vineland Adaptive Behavior Scale (see Adrienne Perry, Helen E Flanagan, Jennifer Dunn Geier, Nancy L Freeman. Brief report: the Vineland Adaptive Behavior Scales in young children with autism spectrum disorders at different cognitive levels. J Aut Dev Disord 2009; 39:1066-1078) or by ADOS scores (see Gotham K, Pickles A, Lord C. Standardizing ADOS scores for a measure of severity in autism spectrum disorders. J Aut Dev Disord 2009; 39:693-705). In other embodiments, the method further comprises determining whether or not the subject will benefit from supplemental vitamin D treatments. This determination can be based on clinically available and research definitions of functional vitamin D status (for example, see Holick, Curr Drug Targets. 2011 January; 12(1):4-18 and Boullata, Curr Opin Clin Nutr Metab Care. 2010 November; 13(6):677-84).

In one aspect, the invention provides for a method for detecting the presence of or a predisposition to a vitamin D deficiency-related neurodevelopmental disorder in a human subject. In some embodiments, the method comprises: (a) obtaining a biological sample from a human subject; (b) detecting whether or not there is an alteration in the expression of a Gc globulin protein in the subject as compared to a non-autistic subject; and (c) measuring vitamin D levels in the subject. In other embodiments, the method comprises: (a) obtaining a biological sample from a human subject; (b) detecting whether or not there is a nucleic acid sequence encoding a Gc globulin protein having the sequence of SEQ ID NO: 2, 4, 6, 8, 9, or 10, or a combination thereof; and (c) measuring vitamin D levels in the subject.

An aspect of the invention is directed to methods for detecting the presence of or a predisposition to a vitamin D deficiency-related immune deficit in a human subject. In some embodiments, the method comprises: (a) obtaining a biological sample from a human subject; (b) detecting whether or not there is an alteration in the expression of a Gc globulin protein in the subject as compared to a non-autistic subject; and (c) measuring vitamin D levels in the subject. In other embodiments, the method comprises: (a) obtaining a biological sample from a human subject; (b) detecting whether or not there is a nucleic acid sequence encoding a Gc globulin protein having the sequence of SEQ ID NO: 2, 4, 6, 8, 9, or 10, or a combination thereof; and (c) measuring vitamin D levels in the subject.

Accordingly, in one aspect, the invention provides a method for treating autism or an autism spectrum disorder (ASD) in a human subject or a child of a human subject. In some embodiments, the method comprises: (a) obtaining a biological sample from a human subject; (b) detecting whether or not there is an alteration in the expression of a Gc globulin protein in the subject as compared to a non-autistic subject; (c) measuring vitamin D levels in the subject; and (d) administering a treatment for autism or an autism spectrum disorder (ASD) if there is an alteration in the expression of a Gc globulin protein in the subject as compared to a non-autistic subject, if the level of vitamin D in the subject is lower than compared to a non-autistic subject, or a combination thereof.

The invention also provides for a diagnostic kit comprising products and reagents for detecting in a sample from a subject the presence of an alteration in a Gc globulin gene (e.g., Gc1F, Gc1s, or Gc2), or a Gc globulin polypeptide polypeptide; alteration in the expression of a Gc globulin gene (e.g., Gc1F, Gc1s, or Gc2); and/or the presence of a Gc globulin polypeptide. The kit can be useful for determining whether a sample from a subject exhibits reduced Gc globulin expression (e.g., Gc1s or Gc2) or exhibits a gene deletion of a Gc globulin. The kit can be useful for determining whether a sample from a subject exhibits increased Gc globulin expression (e.g., Gc1F). For example, the diagnostic kit according to the present invention comprises any primer, any pair of primers, any nucleic acid probe and/or any ligand, (for example, an antibody directed to a Gc globulin). The diagnostic kit according to the present invention can further comprise reagents and/or protocols for performing a hybridization, amplification, or antigen-antibody immune reaction. In one embodiment, the kit can comprise nucleic acid primers that specifically hybridize to and can prime a polymerase reaction from a Gc globulin biomarker (e.g., Gc1F, Gc1s, or Gc2) comprising SEQ ID NOS: 2, 4, 6, 8, 9, or 10, or a combination thereof. In another embodiment, the primer can comprise a nucleotide sequence directed to SEQ ID NOS: 2, 4, 6, 8, 9, or 10. In some embodiments, the kit comprises an antibody that specifically binds to a Gc globulin biomarker comprising SEQ ID NO: 1, 3, 5, 11, 12, or 13, wherein the antibody will recognize the protein only when a Gc globulin biomarker is present

The diagnosis methods can be performed in vitro, ex vivo, or in vivo. These methods utilize a sample from the subject in order to assess the status of a Gc globulin gene locus. The sample can be any biological sample derived from a subject, which contains nucleic acids or polypeptides. Examples of such samples include, but are not limited to, fluids, tissues, cell samples, organs, and tissue biopsies. Non-limiting examples of samples include blood, cord blood, liver, plasma, serum, saliva, urine, or seminal fluid. Pre-natal diagnosis can also be performed by testing fetal cells or placental cells, for instance. Screening of parental samples can also be used to determine risk/likelihood of offspring possessing the germline mutation and/or the Gc polymorphism. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. The nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents, such as probes, primers, or ligands, in order to assess the presence of an altered Gc globulin gene locus. Contacting can be performed in any suitable device, such as a plate, tube, well, or glass. In some embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

Identifying an altered polypeptide, RNA or DNA of a Gc globulin (e.g., Gc1F, Gc1s, or Gc2) in a sample is indicative of the presence of an altered Gc globulin gene in the subject, which can be correlated to the presence, predisposition or stage of progression of autism or an autism spectrum disorder. For example, an individual having a germ line mutation or a polymorphism in a Gc globulin gene (e.g., Gc1F, Gc1s, or Gc2) has an increased risk of developing autism or an autism spectrum disorder. The determination of the presence of an altered gene locus in a subject also allows the design of appropriate therapeutic intervention, which is more effective and customized. Also, this determination at the pre-symptomatic level allows a preventive regimen to be applied.

The invention provides a method for treating or preventing autism or an autism spectrum disorder in a subject. The invention also provides a method for increasing free serum vitamin D levels in a subject in need thereof. In one embodiment, the method comprises administering to the subject in need a therapeutic treatment against autism or an autism spectrum disorder. In another embodiment, the method comprises administering to the subject in need a therapeutic treatment for increasing free serum vitamin D levels, thus altering the proportion of vitamin D that is bound to a Gc globlin, and increasing the levels of free serum vitamin D. The therapeutic treatment can be a drug administration (for example, a pharmaceutical composition comprising a Gc globulin modulating compound). In some embodiments, the methods further comprise administering vitamin D (e.g., 25-hydroxy-vitamin D, 1,25-dihydroxyvitamin D3, cholecalciferol, or a vitamin D analogue) to the subject in need. In another embodiment, the invention provides for a method for treating or preventing autism or an ASD in a subject in need thereof, the method comprising: administering a therapeutic dosage of vitamin D supplementation to subjects in whom the Gc1f isoform of Gc globulin has been demonstrated at any level, or who have been demonstrated to have elevated levels in blood of any type of Gc globulin (Gc1f, Gc1s, Gc2), thereby treating or preventing autism or an ASD. This method applies to both mothers during pregnancy and to the child from birth forward.

Vitamin D and the Gc globulin modulating compound can be administered simultaneously, or sequentially in any order. In one embodiment, the Gc globulin gene targeted can be a Gc1F, Gc1s, Gc2 gene, or a combination thereof. In another embodiment, the Gc globulin modulating compound is an antibody that specifically binds to a Gc globulin protein or a fragment thereof (e.g., a protein comprising SEQ ID NO: 1, 3, 5, 11, 12, 13, 14, 15 or 17); an antisense RNA or antisense DNA directed to SEQ ID NO: 2, 4, 6, 8, 9, 10, 16, or 18 that decreases expression of a Gc globulin polypeptide; a siRNA that specifically targets a Gc globulin gene; or a combination thereof. In some embodiments, the Gc globulin compound comprises a Gc globulin polypeptide of about 10 amino acids comprising at least about 75%, at least about 80%, at least about 85%, at least about 90%, at least about 93%, at least about 95%, at least about 97%, at least about 98%, at least about 99%, or 100% of SEQ ID NO: 1, 3, 5, 11, 12, 13, 14, 15 or 17. In other embodiments, the Gc globulin compound comprises a vector comprising a nucleic acid sequence encoding a Gc globulin polypeptide of about 10 amino acids comprising SEQ ID NO: 2, 4, 6, 8, 9, 10, 16, or 18.

The subject to be treated can be a human (e.g., a pregnant female) or a child thereof. In one embodiment, the subject is of non-caucasian origin. The subject can also be a human embryo, a human fetus, or an unborn human child. In some embodiments, the subject is afflicted with autism or an autism spectrum disorder. In other embodiments, the subject is afflicted with a vitamin D deficiency-related neurodevelopmental disorder. In further embodiments, the subject is afflicted with a vitamin D deficiency-related immune deficit.

The invention further provides methods for the use of a therapeutic amount of GcMAF. In one embodiment, GcMAF is used for treating or preventing autism or an autism spectrum disorder in a subject. In another embodiment, GcMAF is used for treating or preventing a vitamin D deficiency-related neurodevelopmental disorder in a subject. In a further embodiment, GcMAF is used for treating or preventing a vitamin D deficiency-related immune deficit in a subject. The therapeutic amount of GcMAF used is at least about 0.0001 at least about 0.00025 at least about 0.0005 at least about 0.00075 at least about 0.001 at least about 0.0025 at least about 0.005 at least about 0.0075 at least about 0.01 at least about 0.025 at least about 0.05 at least about 0.075 at least about 0.1 at least about 0.25 at least about 0.5 at least about 0.75 at least about 1 at least about 5 or at least about 10 μg. In some embodiments, the methods further comprise administering vitamin D (e.g., 25-hydroxy-vitamin D, colecalciferol, or a vitamin D analogue) to the subject in need. Vitamin D and GcMAF can be administered simultaneously, or sequentially in any order. The subject to be treated can be a human (e.g., a pregnant female) or a child thereof. In one embodiment, the subject is of non-caucasian origin. The subject can also be a human embryo, a human fetus, or an unborn human child.

Administration and Dosing

A Gc globulin molecule (e.g., Gc1F, Gc1s, or Gc2) or a Gc globulin modulating compound can be administered to the subject once (e.g., as a single injection or deposition). Alternatively, a Gc globulin or Gc globulin modulating compound of the invention can be administered once or twice daily to a subject in need thereof for a period of from about two to about twenty-eight days, or from about seven to about ten days. It can also be administered once or twice daily to a subject for a period of 1, 2, 3, 4, 5, 6, 7, 8, 9, 10, 11, 12 times per year, or a combination thereof. Furthermore, the Gc globulin or Gc globulin modulating compound of the invention can be co-administrated with another therapeutic, such as an anti-depressant, an anti-psychotic, a benzodiazepine drug, vitamin D (such as 25-hydroxy-vitamin D, colecalciferol, or a vitamin D analogue), or a combination thereof. Where a dosage regimen comprises multiple administrations, the effective amount of the Gc globulin or Gc globulin modulating compound administered to the subject can comprise the total amount of gene product administered over the entire dosage regimen.

The Gc globulin or Gc globulin modulating compounds of the invention can be administered to a subject by any means suitable for delivering the Gc globulin molecule or Gc globulin modulating compound to cells of the subject. For example, Gc globulin molecule or Gc globulin modulating compound can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with autism or an ASD by any means that produces contact of the active ingredient with the agent's site of action in the body of an animal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's The Science and Practice of Pharamcy, 20^{th} ed. Lippincott Williams & Wilkins, Philadelphia, Pa. (2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Pharmaceutical formulations of the invention can comprise a Gc globulin or Gc globulin modulating compound (e.g., 0.1 to 90% by weight), or a physiologically acceptable salt thereof, mixed with a pharmaceutically-acceptable carrier. The pharmaceutical formulations of the invention can also comprise the Gc globulin or Gc globulin modulating compound of the invention which are encapsulated by liposomes and a pharmaceutically-acceptable carrier. Useful pharmaceutically-acceptable carriers are water, buffered water, normal saline, 0.4% saline, 0.3% glycine, or hyaluronic acid.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, or magnesium carbonate.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, or magnesium carbonate. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, or sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Nucleic acids, peptides, or polypeptides of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix (1996) Pharm Res. 13: 1760-1764; Samanen (1996) J. Pharm. Pharmacol. 48: 119-135; U.S. Pat. No. 5,391,377, describing lipid compositions for oral delivery of therapeutic agents (for example, liposomal delivery). In one embodiment, the Gc globulin molecule (e.g., Gc1F, Gc1s, or Gc2) or Gc globulin modulating compound can be delivered to the alimentary canal or intestine of the subject via oral administration that it can withstand digestion and degradation.

For oral administration, the therapeutic compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active agent. For buccal administration the therapeutic compositions can take the form of tablets or lozenges formulated in a conventional manner. For administration by inhalation, the compositions for use are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflate, can be formulated containing a powder mix of the therapeutic agents and a suitable powder base such as lactose or starch.

The therapeutic compositions can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

Suitable enteral administration routes for the present methods include oral, rectal, or intranasal delivery. Suitable parenteral administration routes include intravascular administration (e.g. intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. For example, Vitamin D, the Gc globulin, and/or Gc globulin modulating compound of the invention can be administered by injection, infusion, and/or oral delivery.

In addition to the formulations described previously, the therapeutic compositions can also be formulated as a depot preparation. Such long acting formulations can be administered by implantation (for example subcutaneously or intramuscularly) or by intramuscular injection. For example, the therapeutic compositions can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, the compositions of the invention are formulated into ointments, salves, gels, or creams as generally known in the art. A wash solution can be used locally to treat an injury or inflammation to accelerate healing. For oral administration, the therapeutic compositions are formulated into conventional oral administration forms such as capsules, tablets, and tonics.

A composition of the present invention can also be formulated as a sustained and/or timed release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the present invention can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions. Single unit dosage forms suitable for oral administration, such as, but not limited to, tablets, capsules, gel-caps, caplets, or powders, that are adapted for sustained release are encompassed by the present invention.

In the present methods, the Gc globulin or Gc globulin modulating compound can be administered to the subject either as RNA, in conjunction with a delivery reagent, or as a nucleic acid (e.g., a recombinant plasmid or viral vector) comprising sequences which expresses the gene product. Suitable delivery reagents for administration of the Gc globulin or Gc globulin modulating compound include the Mirus Transit TKO lipophilic reagent; lipofectin; lipofectamine; cellfectin; or polycations (e.g., polylysine), or liposomes.

The dosage administered can be a therapeutically effective amount of the composition sufficient to result in amelioration of symptoms of autism or an autism spectrum disorder, of a vitamin D deficiency-related neurodevelopmental disorder, or of a vitamin D deficiency-related immune deficit in a subject, and can vary depending upon known factors such as the pharmacodynamic characteristics of the active ingredient and its mode and route of administration; age, sex, health and weight of the recipient; nature and extent of symptoms; kind of concurrent treatment, frequency of treatment and the effect desired.

For example, an effective amount of vitamin D can be administered to a subject in need thereof. The vitamin D dosage can be adjusted according to total and free 25-hydroxy-vitamin D levels as calculated by measurement in blood (plasma or serum) of levels of 25-hydroxy-vitamin D and Gc, or according to direct measurements of free serum 25(OH)D and 1,25(OH)2D through methods such as centrifugal ultrafiltration or equilibrium dialysis; or any combination thereof. In some embodiments, the subject in need thereof is exposed to ultraviolet light in order to synthesize vitamin D. For example, the UVB output of a sun lamp should overlap with the region of UVB necessary for vitamin D production in the skin (e.g., being in the spectral range of 290-315 nm) (see Chandra et al., Photodermatol Photoimmunol Photomed. 2007 October; 23(5):179-85, which is incorporated by reference in its entirety). In one embodiment, the subject is exposed to UV light for sessions of at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 16 minutes, at least 18 minutes, at least 20 minutes, or at least 25 minutes. In another embodiment, the subject attends a session at least once a week, at least 2 times a week, at least 3 times per week, at least 4 times per week, or at least 5 times per week. In a further embodiment, the subject is exposed to a UV light session for at least 3 weeks, at least 4 weeks, at least 5 weeks, at least 6 weeks, at least 8 weeks, at least 12 weeks, or at least 16 weeks.

In one embodiment, the therapeutically effective amount of the administered 25-hydroxy-vitamin D is a concentration of at least about 10 ng/ml, at least about 12.5 ng/ml, at least about 15 ng/ml, at least about 20 ng/ml, at least about 25 ng/ml, at least about 30 ng/ml, at least about 35 ng/ml, at least about 40 ng/ml, 45 ng/ml, at least about 50 ng/ml, at least about 55 ng/ml, at least about 60 ng/ml, at least about 65 ng/ml, at least about 70 ng/ml, or at least about 75 ng/ml.

In another embodiment, 1,25-dihydroxyvitamin D3 can be administered to a subject in need thereof. In one embodiment, 1,25-dihydroxyvitamin D3 is calcitriol. In another embodiment, the therapeutically effective amount of the administered calcitriol is a a concentration of at least about 25 ng, at least about 50 ng, at least about 100 ng, at least about 125 ng, at least about 150 ng, at least about 200 ng, at least about 250 ng, at least about 300 ng, at least about 350 ng, at least about 400 ng, 450 ng, at least about 500 ng, at least about 550 ng, at least about 600 ng, at least about 650 ng, at least about 700 ng, at least about 750 ng, at least about 800 ng, 850 ng, at least about 900 ng, at least about 1000 ng, at least about 1100 ng, at least about 1200 ng, at least about 1300 ng, at least about 1400 ng, or at least about 1500 ng. In some embodiments, wherein calcitriol is administered 1 time per week, 2 times per week, 3 times per week, 4 times per week, or 5 times per week. In other embodiments, calcitriol is adjusted weekly in 250 ng increments. For example, the adjustments can be based on the levels of serum iPTH, serum calcium, and the calcium times phosphate value (obtained twice weekly). Serum calcium, phosphorus, magnesium and alkaline phosphatase and 24-hour urinary calcium and phosphorus should be determined every other week.

The international unit (IU) is a quantity of a biologic (such as a vitamin). One IU is a unit that produces a particular biological effect agreed upon as an international standard. In one embodiment, the therapeutically effective amount of the administered cholecalciferol is at least about 1000 IU, at least about 2000 IU, at least about 2500 IU, at least about 3000 IU, at least about 3500 IU, at least about 4000 IU, at least about 4500 IU, at least about 5000 IU, at least about 5500 IU, at least about 6000 IU, at least about 6500 IU, at least about 7000 IU, at least about 7500 IU, at least about 8000 IU, at least about 8500 IU, at least about 9000 IU, at least about 9500 IU, at least about 10000 IU, at least about 11000 IU, at least about 12000 IU, at least about 13000 IU, at least about 14000 IU, at least about 15000 IU, at least about 16000 IU, at least about 17000 IU, at least about 18000 IU, at least about 19000 IU, at least about 20000 IU, or at least about 25000 IU. In one embodiment, cholecalciferol is administered daily. For example, the cholecalciferol dosage can be adjusted according to total and free 25-hydroxy-vitamin D levels as calculated by measurement in the blood (plasma or serum) of levels of 25-hydroxy-vitamin D and Gc, or according to direct measurements of free serum 25(OH)D and 1,25(OH)2D through methods such as centrifugal ultrafiltration or equilibrium dialysis.

In one embodiment, the therapeutically effective amount of the administered vitamin D analogue (e.g., ergo-colecalciferol) is a concentration of at least about 50000 IU. In some embodiments, ergo-colecalciferol is administered weekly for at least about 1 week, 2 weeks, 4 weeks, 6, weeks, 8 weeks, 16 weeks, 20 weeks, 24 weeks, 32 weeks, 40 weeks, or 52 weeks. For example, the ergo-cholecalciferol dosage is adjusted according to total and free 25-hydroxy-vitamin D levels as calculated by measurement in blood (plasma or serum) of levels of 25-hydroxy-vitamin D and Gc, or according to direct measurements of free serum 25(OH)D and 1,25(OH)2D through methods such as centrifugal ultrafiltration or equilibrium dialysis.

In some embodiments, the effective amount of the administered Gc globulin modulating compound (e.g., a compound described herein that is directed to Gc1F, Gc1s, or Gc2) is at least about 0.0001 μg/kg body weight, at least about 0.00025 μg/kg body weight, at least about 0.0005 μg/kg body weight, at least about 0.00075 μg/kg body weight, at least about 0.001 μg/kg body weight, at least about 0.0025 μg/kg body weight, at least about 0.005 μg/kg body weight, at least about 0.0075 μg/kg body weight, at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10μg/kg body weight, at least about 25 μg/kg body weight, at least about 50μg/kg body weight, at least about 75 μg/kg body weight, at least about 100μg/kg body weight, at least about 150μg/kg body weight, at least about 200μg/kg body weight, at least about 250μg/kg body weight, at least about 300μg/kg body weight, at least about 350μg/kg body weight, at least about 400μg/kg body weight, at least about 450μg/kg body weight, at least about 500μg/kg body weight, at least about 550μg/kg body weight, at least about 600μg/kg body weight, at least about 650μg/kg body weight, at least about 700μg/kg body weight, at least about 750μg/kg body weight, at least about 800μg/kg body weight, at least about 850μg/kg body weight, at least about 900μg/kg body weight, at least about 950μg/kg body weight, at least about 1000μg/kg body weight, at least about 2000μg/kg body weight, at least about 3000μg/kg body weight, at least about 4000μg/kg body weight, at least about 5000μg/kg body weight, at least about 6000μg/kg body weight, at least about 7000 μg/kg body weight, at least about 8000μg/kg body weight, at least about 95000μg/kg body weight, or at least about 10,000μg/kg body weight.

Toxicity and therapeutic efficacy of therapeutic compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are useful. Therapeutic compositions that exhibit some toxic side effects can be used.

A therapeutically effective dose of Gc globulin or Gc globulin modulating compound can depend upon a number of factors known to those or ordinary skill in the art. The dose(s) of the Gc globulin or Gc globulin modulating compound can vary, for example, depending upon the identity, size, and condition of the subject or sample being treated, further depending upon the route by which the composition is to be administered, if applicable, and the effect which the practitioner desires the Gc globulin or Gc globulin modulating compound to have upon the nucleic acid or polypeptide of the invention. These amounts can be readily determined by a skilled artisan.

Pharmaceutical Composition and Therapy

The invention provides methods for treating or preventing autism or an autism spectrum disorder in a subject. In one embodiment, the method can comprise administering to the subject a functional (e.g., wild-type) Gc globulin molecule (e.g., Gc1F, Gc1s, or Gc2) or Gc globulin modulating compound, which can be a polypeptide or a nucleic acid. In one embodiment, the Gc globulin modulating compound is an antibody that specifically binds to a Gc globulin protein or a fragment thereof (e.g., a protein comprising SEQ ID NO: 1, 3, 5, 11, 12, 13, 14, 15, or 17); an antisense RNA or antisense DNA directed to SEQ ID NO: 2, 4, 6, 8, 9, 10, 16, or 18 that decreases expression of a Gc globulin polypeptide; a siRNA that specifically targets a Gc globulin gene (e.g., SEQ ID NO: 2, 4, or 6); a small peptide directed to SEQ ID NO: 1, 3, 5, 11, 12, 13, 14, 15, or 17; or a combination thereof.

Various approaches can be carried out to restore the Gc globulin function in a subject or to establish Gc globulin modulating activity in a subject, such as those carrying an altered gene locus comprising a Gc globulin gene (e.g., Gc1F, Gc1s, or Gc2). For example, supplying a Gc globulin modulating compound to such subjects can suppress phenotypic expression of autism or an autism spectrum disorders in a pathological cell or organism. Decreasing Gc globulin activity or expression can be accomplished through gene or protein therapy as discussed herein.

In one embodiment, a nucleic acid encoding a Gc globulin or a functional part thereof can be introduced into the cells of a subject. In another embodiment, a nucleic acid encoding a Gc globulin modulating compound can be introduced into the cells of a subject. The wild-type Gc globulin gene (or a functional part thereof) or Gc globulin modulating compound can also be introduced into the cells of the subject in need thereof using a vector as described herein. The vector can be a viral vector or a plasmid. The Gc globulin gene or Gc globulin modulating compound can also be introduced as naked DNA. For example, Gc globulin gene or Gc globulin modulating compound can be provided so as to integrate into the genome of the recipient host cells, or to remain extra-chromosomal. Integration can occur randomly or at precisely defined sites, such as through homologous recombination. For example, a functional copy of the Gc globulin gene can be inserted in replacement of an altered version in a cell, through homologous recombination. Further techniques include gene gun, liposome-mediated transfection, or cationic lipid-mediated transfection. Gene therapy can be accomplished by direct gene injection, or by administering ex vivo prepared genetically modified cells expressing a functional polypeptide.

Gene Therapy and Protein Replacement Methods

Delivery of nucleic acids into viable cells can be effected ex vivo, in situ, or in vivo by use of vectors, and more particularly viral vectors (e.g., lentivirus, adenovirus, adeno-associated virus, or a retrovirus), or ex vivo by use of physical DNA transfer methods (e.g., liposomes or chemical treatments). Non-limiting techniques for the transfer of nucleic acid into mammalian cells in vitro include the use of liposomes, electroporation, microinjection, cell fusion, DEAE-dextran, and the calcium phosphate precipitation method (see, for example, Anderson (1998) Nature, 392(6679):25-20). Introduction of a nucleic acid or a gene encoding a polypeptide can also be accomplished with extrachromosomal substrates (transient expression) or artificial chromosomes (stable expression). Cells can also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., 1992), adenovirus (Berkner, 1992; Berkner et al., 1988; Gorziglia and Kapikian, 1992; Quantin et al., 1992; Rosenfeld et al., 1992; Wilkinson et al., 1992; Stratford-Perricaudet et al., 1990), vaccinia virus (Moss, 1992), adeno-associated virus (Muzyczka, 1992; Ohi et al., 1990), herpesviruses including HSV and EBV (Margolskee, 1992; Johnson et al., 1992; Fink et al., 1992; Breakfield and Geller, 1987; Freese et al., 1990), and retroviruses of avian (Biandyopadhyay and Temin, 1984; Petropoulos et al., 1992), murine (Miller, 1992; Miller et al., 1985; Sorge et al., 1984; Mann and Baltimore, 1985; Miller et al., 1988), and human origin (Shimada et al., 1991; Helseth et al., 1990; Page et al., 1990; Buchschacher and Panganiban, 1992). Non-limiting examples of in vivo gene transfer techniques include transfection with viral (typically retroviral) vectors (see U.S. Pat. No. 5,252,479, which is incorporated by reference in its entirety) and viral coat protein-liposome mediated transfection (Dzau et al., (1993) Trends in Biotechnology 11:205-210, incorporated entirely by reference). For example, naked DNA vaccines are generally known in the art; see Brower (1998) Nat. Biotech., 16:1304-1305, which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al. (1994) PNAS 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al. (1992) Science 256:808-813; U.S. Pat. Nos. 5,252,479, 5,747,469, 6,017,524, 6,143,290, 6,410,010 6,511,847; and U.S. Application Publication Nos. 2002/0077313 and 2002/00069, which are all hereby incorporated by reference in their entireties. For additional reviews of gene therapy technology, see Friedmann (1989) Science, 244:1275-1281; Verma, Scientific American: 68-84 (1990); Miller (1992) Nature, 357: 455-460; Kikuchi et al. (2008) J Dermatol Sci. 50(2):87-98; Isaka et al. (2007) Expert Opin Drug Deliv. 4(5):561-71; Jager et al. (2007) Curr Gene Ther. 7(4):272-83; Waehler et al. (2007) Nat Rev Genet. 8(8):573-87; Jensen et al. (2007) Ann Med. 39(2):108-15; Herweijer et al. (2007) Gene Ther. 14(2):99-107; Eliyahu et al. (2005) Molecules 10(1):34-64; and Altaras et al. (2005) Adv Biochem Eng Biotechnol. 99:193-260, all of which are hereby incorporated by reference in their entireties.

Protein replacement therapy can increase the amount of protein by exogenously introducing wild-type or biologically functional protein by way of infusion. A replacement polypeptide can be synthesized according to known chemical techniques or can be produced and purified via known molecular biological techniques. Protein replacement therapy has been developed for various disorders. For example, a wild-type protein can be purified from a recombinant cellular expression system (e.g., mammalian cells or insect cells, see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

A polypeptide encoded by a Gc globulin gene (e.g., Gc1F, Gc1s, or Gc2) or a Gc globulin modulating compound can also be delivered in a controlled release system. For example, the polypeptide and molecule can be administered using intravenous infusion, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science (1990) 249:1527-1533).

These methods described herein are by no means all-inclusive, and further methods to suit the specific application is understood by the ordinary skilled artisan. Moreover, the effective amount of the compositions can be further approximated through analogy to compounds known to exert the desired effect.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Vitamin D Binding Protein Genes and Gene Products as a Biomarker for Autism

Autism spectrum disorders (ASD) comprise a set of severe, disabling conditions that begin in early life and for which causes and cures remain elusive. Early identification of children at highest risk provides the best opportunity for children to derive benefit from interventions, while central nervous system plasticity remains at a maximum.

To identify candidate biomarkers for autism that will assist such early identification and drive discovery of causal factors that might aid in development of preventive therapeutics, umbilical cord blood plasma samples from children diagnosed with autism (n=11 cases) and children without evidence of developmental disorder (n=12 controls) were subjected to proteomic analysis. Participating children were screened and clinically assessed to establish neuropsychiatric diagnosis (or lack thereof) at age 3 years, and their previously stored umbilical cord blood (birth) samples were obtained. Subjects for this analysis were selected from among a larger set of 81 samples representing the key diagnostic group, autism; comparator diagnostic groups (language disorder, cerebral palsy, low birth weight); and typically developing control children.

Proteomic assays were pursued. Cord blood plasma samples were pooled according to diagnosis and sex (6 male and 5 female autism cases; 6 male and 6 female controls) and then depleted of the most abundant plasma proteins by affinity chromatography (IgY 14 column). Cord blood plasma samples from 10 additional female control subjects were split into two additional pools, each comprised of five cord blood plasma samples. Following depletion and amino acid analyses of all sample plasma pools to ensure that equivalent concentrations remained of the remaining amino acid content, samples were analyzed by Differential Gel Electrophoresis (DIGE) and multiplexed Isobaric Tagging technology for Relative and Absolute Quantitation (iTRAQ, run as an 8-plex assay comparing 2 aliquots from each of the following sample pools: male autism cases, female autism cases, male control subjects, female control subjects). After establishing the suitability of these cord blood plasma samples for proteomic analysis, subsequent comparison of cases and controls revealed differential expression of markers of inflammation and immune disturbance, oxidative stress and xenobiotic metabolism, and endocrine pathways, including, but not limited to, vitamin D metabolism. Using iTRAQ, an increase in autism cases was found as compared with control subjects of the precursor protein for group-specific component ([Gc], also known as Gc globulin or vitamin D binding protein), the major plasma carrier protein of vitamin D and its metabolites, (males, p=0.0005; females, p=0.000003). Gc was also elevated by DIGE in autism male and autism female sample pools relative to control pools).

iTRAQ profiles were accompanied by different post-translational isoforms of Gc-related peptides, suggesting the presence of multiple genetic variants of Gc in the sample set. Quantitative analysis of group differences in Gc tryptic peptides was performed by Multiple Reaction Monitoring (MRM) to distinguish the 3 most common electrophoretic Gc variants. The increase in Gc in the autism sample pool is largely explained by a 2.5-to 2.8-fold increase in the level of peptides associated with Gc1F, a Gc variant rare among European/Scandinavian individuals, but reported most frequently in dark—or yellow—skinned populations (Chun et al., 2010). Three major polymorphic variants of Gc have been described: Gc1F variant, arising from the ancestral Gc1F allele; and two subsequent variants, Gc1S and Gc2, each representing a single amino acid change in Gc1F (D416E for Gc1S; T420K for Gc2).

These polymorphisms affect the affinity of Gc for vitamin D ligands (Arnaud and Constans, 1993) as well as Gc serum levels (Lauridsen et al., 2001), with Gc1F having the greatest affinity and abundance, and Gc2 the lowest affinity and abundance. The higher affinity and abundance of Gc1F implies that at any given level of vitamin D intake or sun exposure, there will be lower free 25-hydroxy-vitamin D3 levels available in the peripheral blood to carry out essential functions, including endocrine/intracrine and innate immune/antimicrobial actions. Insufficiency of vitamin D is associated with a wide range of adverse health outcomes, including infectious and autoimmune diseases (Shoenfeld et al. 2009; Chun et al., 2010). Dark-skinned, African populations residing in the US or Europe are not only frequently vitamin D-deficient; they also are more susceptible to certain infections, including tuberculosis, as well as more aggressive forms of intracellular infections (Holick 2007). Without being bound by theory, polymorphisms of the vitamin D binding protein gene and their influence on circulating (free) vitamin D3 levels can mediate the frequently reported findings in autism of immune dysfunction, autoimmunity and association with pre/perinatal infections. These birth biomarker findings, if substantiated in a larger sample, may provide mechanistic insight into reports that autism prevalence is up to 10× higher in the children of dark-skinned (especially Somalian) immigrants to northern latitudes (Scandinavia, Minn.; Barnevik-Olsson et al. 2008; Bejerot & Humble Tidsskr Nor Laegeforen 2008; 128:1986-7; NY Times 2009; McFadden Toxicology 1996; 111:43-65; and Fraser et al., Environ Health Perspect 2009; 117:1520-5). In addition to regulating vitamin D levels, Gc has roles in actin scavenging; fatty acid transport; chemotaxis; and, perhaps most significant for autism pathogenesis, macrophage activation (Gc serves as a precursor of the inflammation-primed macrophage activating factor, GcMAF) and chemotaxis (Nagasawa H, et al. Anticancer Res 2004; 24:3361-6; Nagasawa H, et al. Anticancer Res 2005; 25:3689-95). Individuals homozygous for the Gc1f polymorphism, for example, have the highest GcMAF precursor activity ((Nagasawa H, et al. Anticancer Res 2004; 24:3361-6). Analysis of maternal Gc polymorphisms and levels, assessment of birth 25(OH)D₃ levels, vitamin D intake, and larger sample numbers will be examined.

### Example 2—Gc1f Findings

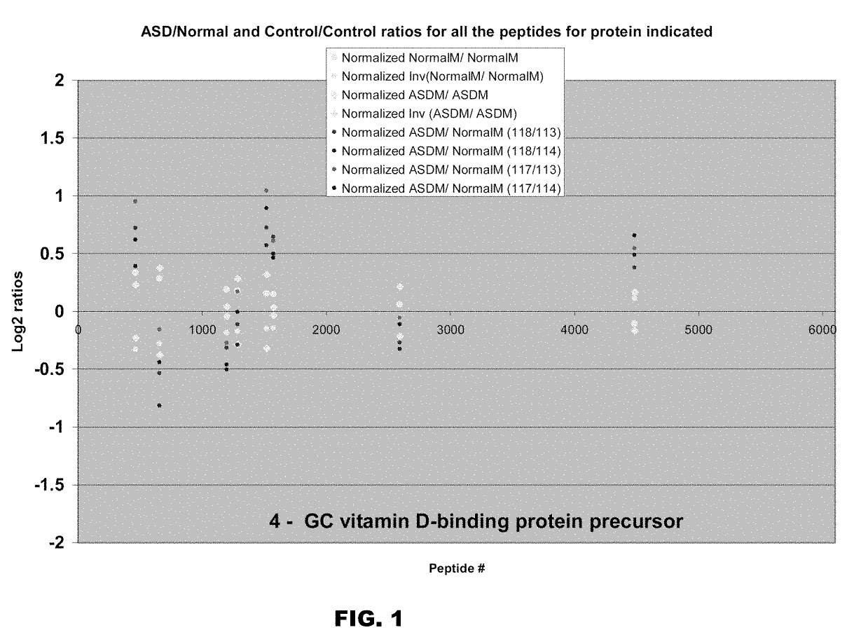

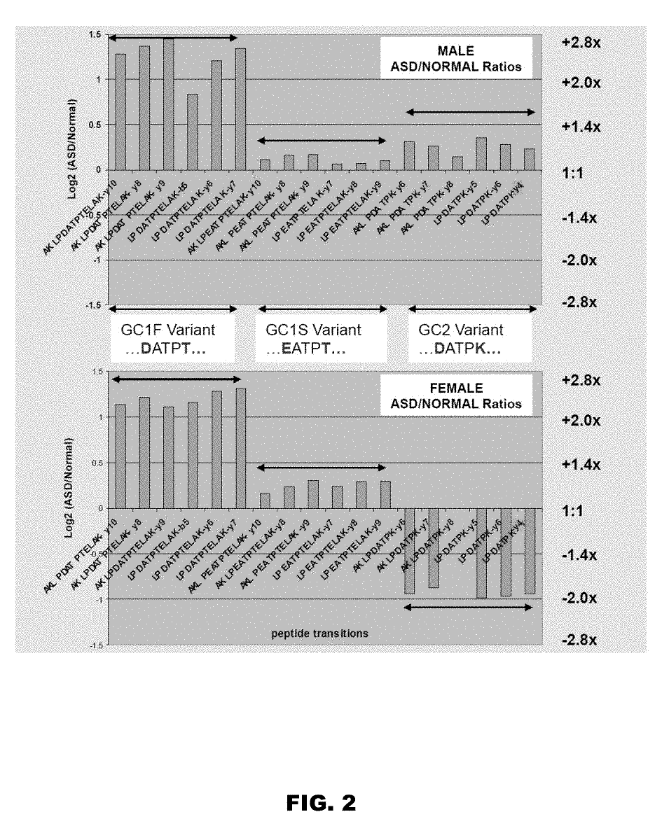

Significantly altered concentrations in the Gc vitamin D-binding protein precursor were previously reported in male and female cases with ASD (male ASD to male control ratios, FIG. 1), identified both with 2DGE and 8-plex iTRAQ. Further examination of specific peptides associated with Gc variants using Multiple Reaction Monitoring (MRM) identified differences in Gc variants in cord blood plasma pools of ASD males and females relative to their sex-matched controls. Gc1f, a variant most common in dark- and yellow-skinned individuals, and relatively rare in the Norwegian population relative to the two other common polymorphisms, Gc1s and Gc2, was nearly 3-fold higher in the cord blood plasma of male and female children with ASD relative to male and female controls (FIG. 2).

Gc1F has the highest avidity for vitamin D, potentially reducing free vitamin D levels by binding vitamin D more tightly. Some studies also find that the Gc1F variant is associated with higher Gc concentrations; higher Gc concentrations would make more binding sites available for any vitamin D that entered the peripheral blood, reducing the free form in the circulation. Without being bound by theory, these factors in concert may act to decrease free vitamin D levels in individuals with the Gc1F allele relative to those with the Gc1s or Gc2 alleles.

Vitamin D binding protein in its deglycosylated form is a macrophage activating factor (GcMAF). It is likely that differences in glycosylation status across the three major Gc variants have an influence on GcMAF activity. Thus, reports of altered innate and adaptive immunity in children with ASD may be influenced by Gc variants as well as plasma levels of free vitamin D.

The Gc1F variant is also associated with diminished induction by 25-OH-vitamin D of cathelicidin, an antimicrobial factor (See Chun et al. J Clin Endocrinol Metab 2010; 95:3368-3376), explaining the associations of Gc1F with greater risk of tuberculosis and other intracellular infections. Some studies suggest that children with ASD are at particular risk of infection by intracellular pathogens.

In addition to reports of differences in Gc levels as a function of Gc variant, Gc concentrations are increased by estrogen (Daiger et al., 1984). Without being bound by theory, alterations in sex hormones may interact with Gc differences to alter risk for ASD. Additionally, a meta-analysis by Dealberto et al. (2011) reported that black ethnicity was associated with an increased risk for autism, a risk that was especially high for autistic disorder as compared with other ASD diagnoses, and interpreted these data as support for the hypothesis that maternal vitamin D insufficiency is a risk factor for autism.

A direct role for vitamin D binding protein (Gc-globulin) in CNS is indicated by reports of the intrathecal synthesis of actin-free Gc-globulin. Without being bound by theory, the differences in Gc variants may also influence the capacity for and avidity of binding of vitamin D in brain, and thus the availability of vitamin D for CNS functions. However, if the behavior of Gc in CNS is similar to that in the peripheral blood, the presence of the Gc1F variant may lead to central vitamin D deficiency. Reduced availability of free Gc in brain may impair the ability to curb inflammation or activated innate immune responses.

Gc and its variants, and other putative biomarkers identified by DIGE and/or iTRAQ, are being further tested.

To define the assay conditions required for quantitation of the levels of Gc and its variant peptides by MRM, and to minimize the influence of experimental variations on these measurements, a labeling technique was used based on isotope-coded affinity tags (ICAT) (Gygi et al., 1999). A series of 12 stable isotope-labeled peptides capable of distinguishing among the three major Gc polymorphisms were labeled by ICAT after optimizing ionization and fragmentation efficiencies for transitions of the parent ions associated with the three primary Gc variants and then validating each observed transition by individual MS/MS. An algorithm for identification and analysis of ICAT-labeled peak pairs in high-resolution LC-MS data that relies on the ratio estimation value of the identified ICAT peak pairs instead of their peak intensity values was used to improve efficiency and accuracy in triggering tandem MS/MS scanning parameters (Yu et al., 2007).

To pilot capacity and conditions for quantitating and differentiating among Gc variants using LC-MS/MS MRM methods, an aliquot derived from the pool of ASD male cord blood plasma that had previously been depleted by passage over a IgY-12 column and used in the initial 2DGE and 8-plex iTRAQ experiments was subjected to high-resolution LC-MS using the ICAT-labeled peptide set. Results of these LC-MS studies with the depleted ASD male cord blood plasma pool sample confirmed that the Gc vitamin D binding protein precursor and its 3 primary variants could be detected, quantitated, and differentiated with high-resolution LC-MS and ICAT-labeled peptides specifically designed to differentiate Gc variants.

To validate the findings of increased levels of Gc, particularly that of the variant found to be elevated in ASD males and females and that is known to have a very low prevalence in the general Norwegian populations, Gc1f, an MRM assay is being developed suitable for use with individual samples. Anticipating a difference in mean Gc levels of about 0.2 and a standard devation of 0.12, and equal sample sizes, the sample size required for an alpha of 0.001 and a power of 99% is about 26 subjects per group. Given the need to conserve the limited volume of samples in this prospective birth cohort study, it was investigated whether sensitive detection of Gc and Gc variants was possible in MRM without prior depletion of samples. If depletion with IgY-12 columns were not required, the time and expense of running individual samples over IgY columns prior to MRM quantitation would be avoided and the volume of irreplaceable samples required for completion of assays would be minimized. A new pool of undepleted cord blood plasma samples was created representing the same ASD males in the pool that had been depleted prior to 2DGE and 8-plex iTRAQ, by combining aliquots of the original (undepleted) individual samples that made up the original pool in equal proportions. After trypsin digestion, the cord blood plasma pool comprised of undepleted samples from ASD males was run using high-resolution LC-MS (Micromass-PC 5600) and analyzed using the SwissProt_2011_10.fasta tax: Homo sapiens (human) database and the MASCOT search engine to define peptide matches above the identity threshold, or above the homology or the identity threshold, controlling for false discovery rate. These studies demonstrated the capacity to detect Gc and its variants in 90-minute LC-MS runs of undepleted samples (protein score, 611; 65.8% coverage; expectation, 1.6E-57). To confirm the reliability of Gc detection using undepleted sample pools in high-resolution LC-MS, and by extension, in MRM, a second, 90-minute run of high-resolution LC-MS was performed on the undepleted ASD male sample pool and analyzed using the same search engine and database. Gc levels determined in this replicate run were nearly identical to those performed in the first run (protein score, 611; 65.8% coverage; expectation, 1.6E-57).

Using the results of protein matches made on the basis of the SwissProt_2011_10.fasta tax: Homo sapiens (human) database, and the identification of peptides associated with those proteins, we are selecting a prioritized set of approximately 500 peptides (100 proteins; 5 peptides per protein), including those representing Gc and Gc variants, to be monitored in MRM LC-MS studies. Quantitative MRM analyses are being designed, targeting the 2-5 peptides best representing each candidate protein biomarker. MRM assay runs are being optimized to monitor already-observed, tryptic peptides on triple quadropole mass spectrometers by extending runs to 3 hours each from 90 minutes; these longer assay runs will help to achieve optimal capacity for distinguishing individual peptides of interest from one another. Undepleted sample pools representing each of the 4 groups used in 2DGE and 8-plex iTRAQ-ASD males, ASD females, control males, control females—will be run for 3 hours each in MRM LC-MS with Gc-targeted, ICAT-labeled peptides. Differences in the levels of Gc and Gc variants across the 4 groups will be examined for statistical significance.

Once refinements required to optimize assay performance for the detection of Gc and its variants are complete, individual samples from 26 ASD cases (15 with mental retardation, and 11 without) and 26 controls will be run over 3 hours each in MRM LC-MS/MS assays with ICAT-labeled, Gc-targeted peptides to enhance efficiency and accuracy in measurements.

### Example 3—Higher Levels of Gc1f Correlate with ASD

New data from MRM-based validation studies provide evidence consistent with the finding described herein that Gc1f is present at higher levels in children with ASD as compared with birth cohort controls. Results of studies using 34 individual undepleted samples from 4 groups (Table 2), run for 3 hours each in MRM LC-MS/MS assays with ICAT-labeled Gc-targeted peptides, indicate a higher ASD/Control log₂-fold change for the Gc1f peptide sequence LPDATPTELAK (SEQ ID NO: 19) (p=0.046; Table 3). Specificity of the Gc1f finding is confirmed by the observation that there was no difference between groups in the relative ASD/Control ratio for Gc2- or Gc1s-associated sequences or in the ratio for conserved Gc sequences present in all three Gc isoforms (P02774-3 sequences, p=ns; also Table 3). Data provided are restricted to those derived from samples with results that demonstrated consistency across 3 isotopes, with isotope dot-products >0.9. Vitamin D is essential for brain development. Given that Gc1f has higher affinity for vitamin D than other vitamin D binding protein isoforms, the presence of higher levels of Gc1f reflects lower levels of the free (unbound), active form of vitamin D in the blood and may be important not only as a biomarker for ASD but also as a potential guide to intervention. Because individuals with high levels of Gc1f are likely to have lower levels of free vitamin D, they may benefit from vitamin D supplementation.

TABLE 2
Proteomics biomarker validation study using MRM.

| | |
|---|---|
| ASD: | 12 males, 6 females |
| CONTROL: | 10 males, 6 females |

All clinic-assessed ~40-43 months of age (including controls)
High rate of mental retardation in ASD group
Similar distribution of month/season and year of birth

TABLE 3
ASD subjects have higher
expression of Gc1f relative to controls.

| Gc | Peptide | ASD/Control | p |
|---|---|---|---|
| type | sequence | Log2 fold change | value |
| Gc2 | AKLPDATPK | 0.88 | 0.491 |
| | (SEQ ID NO: 22) | | |
| Gc1s | LPEATPTELAK | 1.04 | 0.910 |
| | (SEQ ID NO: 21) | | |
| Gc1f | LPDATPTELAK | 1.58 | 0.046 |
| | (SEQ ID NO: 19) | | |
| P02774-3 | EDFTSLSLVLYSR | 1.16 | 0.594 |
| | (SEQ ID NO: 27) | | |
| P02774-3 | RTHLPEVFLSK | 1.01 | 0.936 |
| | (SEQ ID NO: 28) | | |

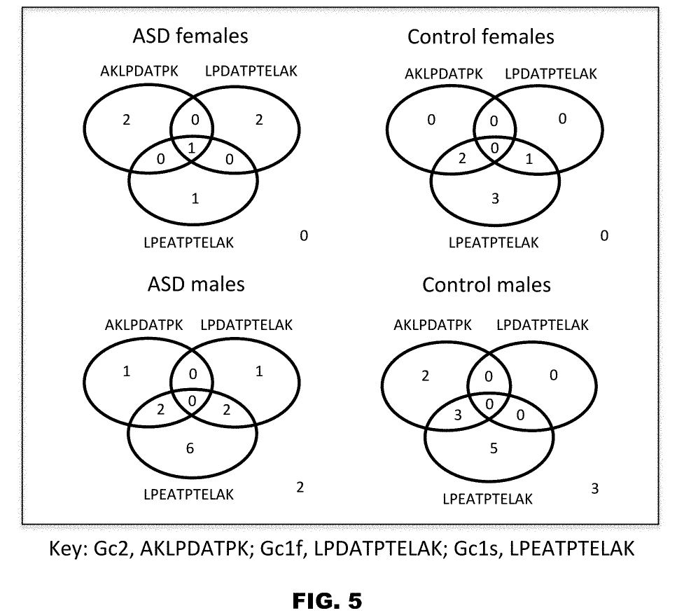

As shown in Table 4, the proportion of subjects with isotope dot-products >0.9 for each of the three major Gc variants (Gc2, Gc1s, Gc1f) shows a trend toward a higher prevalence in the ASD group (p=0.061). Although there were no significant differences when stratified by sex, none of the 10 male controls had any Gc1f sequences (FIG. 5).

TABLE 4
ASD subjects are more likely to have Gc1f
sequences present relative to controls.

| | | | p value |
|---|---|---|---|
| | ASD | Control | (Chi-Square, Fisher's |
| Gc type | n (%) | n (%) | Exact Test, 1-sided) |
| Gc2 | 6 (33.3) | 7 (43.8) | 0.393 |
| Gc1s | 12 (66.7) | 14 (87.5) | 0.153 |
| Gc1f | 6 (33.3) | 1 (6.3) | 0.061 |

Accurate determination of vitamin D status requires determination of Gc globulin variant type and Gc globulin levels in addition to measurement of vitamin D levels.

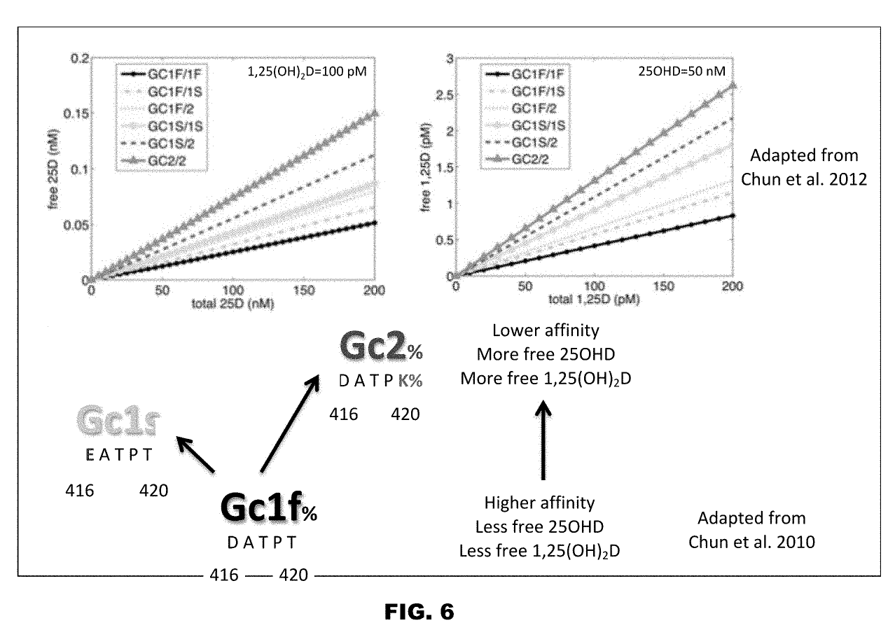

Assessments of vitamin D status that fail to take Gc globulin variant types and levels into account can lead to serious overestimates of true vitamin D status and missed cases of vitamin D deficiency. As shown in FIG. 6, free vitamin D (both mono- and di-hydroxy forms) is significantly lower in individuals with the Gc1f variant type, with lowest levels in individuals homozygous for Gc1f. Free vitamin D status is also lower in individuals with the Gc1s type compared to individuals possessing the Gc2 variant. Determination of Gc globulin variant type and measurement of levels of Gc globulin as per methods described herein are essential for accurate assessment of vitamin D status. In some embodiments, measurements of vitamin D status include determination of Gc globulin variant type, analysis of levels of Gc globulin as well as measurements of total and free vitamin D levels. In some embodiments, analysis of vitamin D levels (vitamin D3, 25-hydroxyvitamin D3 and/or 24,25-dihydroxyvitamin D3) can be performed by LC-MS/MS targeting vitamin D and its metabolites (Burild et al. 2014). In other embodiments, total vitamin D levels (vitamin D3, 25-hydroxyvitamin D3 and/or 24,25-dihydroxyvitamin D3) can be measured by immunoassays including ELISA using an antibody directed to vitamin D; western blot using an antibody directed to vitamin D; or bead-based immunoassay available commercially, such as from Luminex or other related detection platforms. Detection can be performed in samples from maternal and child ASD and control pairs as well as in samples from fathers (mothers during both pregnancy and at birth; children at birth and at later time points, including first diagnosis; fathers prior to or at conception or at later time points). Determination of both maternal and paternal Gc globulin variant type prior to conception or early in pregnancy can allow for early identification of pregnancies that may require closer and more accurate monitoring of vitamin D status. Season of the year at conception and birth, estimated UV exposures based on geographic location of residence as well as estimated, prospectively ascertained vitamin D intake during pregnancy and early postnatal life (including breastfeeding) can also be determined.

### REFERENCES FOR EXAMPLES 1-3

Burild A, Frandsen H L, Jakobsen J. Simultaneous quantification of vitamin D3, 25-hydroxyvitamin D3 and 24,25-dihydroxyvitamin D3 in human serum by LC-MS/MS. Scand J Clin Lab Invest. 2014; 74:418-423
Chun R F, Lauridsen A L, Suon L, Zella L A, Pike J W, Modlin R L, Martineau A R, Wilkinson R J, Adams J, Hewison M. Vitamin D-binding protein directs monocyte responses to 25-hydroxy- and 1,25-dihydroxyvitamin D. J Clin Endocrinol Metab 2010; 95:3368-3376.
Chun R F, Peercy B E, Adams J S, Hewison M. Vitamin D binding protein and monocyte response to 25-hydroxyvitamin D and 1,25-dihydroxyvitamin D: analysis by mathematical modeling. PLoS One. 2012; 7:e30773.
Daiger S P, Miller M, Chakraborty R. Heritability of quantitative variation at the group-specific component (Gc) locus. Am J Hum Genet. 1984; 36:663-676
Dealberto M J. Prevalence of autism according to maternal immigrant status and ethnic origin. Acta Psychiatr Scand. 2011; 123:339-348
Gygi S, Rist B, Gerber S, Turecek F, Gelb M, Aebersold R. Quantitative analysis of complex protein mixtures using isotope-coded affinity tags. Nat Biotechnol. 1999; 17:994-999
Yu W, Liu J, Colangelo C, Gulcicek E, Zhao H. A new protocol of analyzing isotope-coded affinity tag data from high-resolution LC-MS spectrometry. Comput Biol Chem. 2007; 31:215-221

1-71. (canceled)

72. A method of treating or preventing autism or an autism spectrum disorder (ASD) in a subject in need thereof, the method comprising administering to the subject a therapeutic amount of a pharmaceutical composition comprising a Gc globulin modulating compound, thereby treating or preventing autism or an ASD.

73. A method for increasing free serum vitamin D levels of a subject in need thereof, the method comprising administering to the subject a therapeutic amount of a pharmaceutical composition comprising a Gc globulin modulating compound, thereby altering the proportion of vitamin D that is bound to Gc globlin, and increasing the levels of free serum vitamin D.

74. The method of claim 73, wherein the subject is afflicted with autism, an autism spectrum disorder, a vitamin D deficiency-related neurodevelopmental disorder, or a vitamin D deficiency-related immune deficit.

75. The method of claim 72, wherein the Gc globulin modulating compound comprises an antibody that specifically binds to a Gc globulin protein or a fragment thereof; an antisense RNA or antisense DNA that decreases expression of a Gc globulin protein; a siRNA that specifically targets a Gc globulin gene; or a combination thereof.

76. The method of claim 72, wherein the Gc globulin modulating compound comprises an antibody that specifically binds to an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof.

77. The method of claim 72, wherein the Gc globulin modulating compound comprises a peptide comprising at least about 10 amino acids of SEQ ID NO: 1, 3, 5, 11, 12, or 13 or a vector comprising a nucleic acid sequence comprising SEQ ID NO: 2, 4, 6, 8, 9, or 10.

78. The method of claim 72, further comprising measuring the Gc globulin level in a sample from the subject before administration and wherein the Gc globulin modulating compound is administered to the subject if the level of Gc globulin in the sample from the subject is increased compared to the level of Gc globulin in a sample from a healthy subject.

79. The method of claim 78, wherein the level of Gc globulin is the level of Gc1s globulin, Gc2 globulin, Gc1f globulin, or a combination thereof.

80. The method of claim 78, wherein the level of Gc globulin is the level of Gc1f globulin.

81. The method of claim 78, wherein the Gc globulin level is the level of Gc globulin protein or the level of Gc globulin nucleic acid.

82. The method of claim 81, wherein the Gc globulin nucleic acid is a nucleic acid sequence encoding a Gc globulin protein having the sequence of SEQ ID NO: 2, 4, 6, 8, 9, or 10.

83. The method of claim 78, wherein the Gc globulin level is measured by: northern blot; real time PCR and primers directed to SEQ ID NO: 2, 4, 6, 8, 9, or 10; a ribonuclease protection assay; a hybridization, amplification, or sequencing technique to distinguish SEQ ID NOs: 2, 4, 6, 8, 9, or 10; ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NOs: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof.

84. A method for detecting the presence of or a predisposition to autism, an autism spectrum disorder (ASD), a vitamin D deficiency-related neurodevelopmental disorder, or a vitamin D deficiency-related immune deficit in a human subject, the method comprising:
measuring the level of Gc globulin in a sample from the subject wherein the Gc globulin level is measured by: northern blot; real time PCR and primers directed to SEQ ID NOs: 2, 4, 6, 8, 9, or 10; a ribonuclease protection assay; a hybridization, amplification, or sequencing technique to distinguish SEQ ID NOs: 2, 4, 6, 8, 9, or 10; ELISA using an antibody directed to SEQ ID NO: 1, 3, 5, 11, 12, or 13; western blot using an antibody directed to SEQ ID NO: 1, 3, 5, 11, 12, or 13; mass spectroscopy, isoelectric focusing, or electrophoresis-based techniques targeting epitopes of SEQ ID NO: 1, 3, 5, 11, 12, or 13; mass spectroscopy, isoelectric focusing, or electrophoresis-based techniques targeting the glycosylation or sialylation status of actin-complexed Gc globulin protein; mass spectroscopy, isoelectric focusing, or electrophoresis-based techniques targeting the glycosylation or sialylation status of actin-free Gc globulin protein; or a combination thereof; and wherein the subject is diagnosed with autism, an autism spectrum disorder (ASD), a vitamin D deficiency-related neurodevelopmental disorder, or a vitamin D deficiency-related immune deficit if the level of Gc globulin is increased compared to the level of Gc globulin in a sample from a healthy subject.

85. A composition for elevating serum vitamin D levels in a subject, the composition in an admixture of a pharmaceutically acceptable carrier comprising a Gc globulin modulating compound.

86. The composition of claim 85, wherein the compound comprises an antibody that specifically binds to a Gc globulin protein or a fragment thereof; an antisense RNA or antisense DNA that decreases expression of a Gc globulin polypeptide; a siRNA that specifically targets a Gc globulin gene; or a combination thereof.

87. The composition of claim 86, wherein the Gc globulin protein comprises an actin-complexed Gc globulin; an actin-free Gc globulin; a glycosylated actin-complexed Gc globulin; a glycosylated actin-free Gc globulin; a sialylated actin-complexed Gc globulin; a sialylated actin-free Gc globulin; or a combination thereof.

88. The composition of claim 85, wherein the compound comprises a peptide comprising at least about 10 amino acids of SEQ ID NO: 1, 3, 5, 11, 12, or 13 or a vector comprising a nucleic acid sequence comprising SEQ ID NO: 2, 4, 6, 8, 9, or 10.

89. A method for treating or preventing autism, an autism spectrum disorder (ASD), a vitamin D deficiency-related neurodevelopmental disorder, or a vitamin D deficiency-related immune deficit in a subject in need thereof, the method comprising:
a) administering to the subject a therapeutic amount of GcMAF,
thereby treating or preventing autism or an ASD.

90. The method of claim 89, wherein the vitamin D deficiency-related neurodevelopmental disorder comprises an increase in volume of lateral ventricles, a decrease in brain cortex thickness, a decrease in neurite outgrowth, an increase in dentate gyrus mitotic cells, an increase in basal ganglia mitotic cells, an increase in hypothalamus mitotic cells, a decrease in calcium uptake, a decrease in dentate gyrus apoptotic cells, a decrease in basal ganglia apoptotic cells, a decrease in hypothalamus apoptotic cells, an increase in subventricular zone (SVZ) neurospheres, a decrease in levels of nerve growth factor (NGF), a decrease in levels of Glial cell-derived neurotrophic factor (GDNF), a decrease in levels of nurr1 transcription factor, a decrease in levels of p75 neurotrophin receptor (p75NTR), a decrease in levels of catechol-O-methyltransferase (COMT), a decrease in levels of neurotrophin-3 (NT-3), a decrease in levels of neurotrophin-4 (NT-4), or a combination thereof.

91. The method of claim 89, wherein the vitamin D deficiency-related immune deficit comprises type I diabetes mellitus, multiple sclerosis, rheumatoid arthritis, systemic lupus erythematosus, inflammatory bowel disease, asthma, a bacterial infection, a viral infection, or a combination thereof.


SEQUENCE LISTING (28 sequences)

SEQ ID NO: 1 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 2 - DNA, 1895 bp, Homo sapiens
actgctcattcttcagccactagtgctggacagtcccctagcttctcttacactgcatacctgtgtctgagtactcttttcatcagtcactcagccagggtctgcagcatgaacccagcagctggtgtcccctatgaggaactcagcaacagattgcgacagaacccttgagaacaaaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaaaaaaaaaaaaaaaaaaaaaaaaaaaaaaaaaa

SEQ ID NO: 3 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPEATPTELAKLVNKHSDFASNCCSINSPPLYCDSEIDAELKNIL

SEQ ID NO: 4 - DNA, 1895 bp, Homo sapiens
actgctcattcttcagccactagtgctggacagtcccctagcttctcttacactgcatacctgtgtctgagtactcttttcatcagtcactcagccagggtctgcagcatgaacccagcagctggtgtcccctatgaggaactcagcaacagattgcgacagaacccttgagaacaaaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgaggccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaaaaaaaaaaaaaaaaaaaaaaaaaaaaaaaaaa

SEQ ID NO: 5 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDAKPTELAKLVNKHSDFASNCCSINSPPLYCDSEIDAELKNIL

SEQ ID NO: 6 - DNA, 1895 bp, Homo sapiens
actgctcattcttcagccactagtgctggacagtcccctagcttctcttacactgcatacctgtgtctgagtactcttttcatcagtcactcagccagggtctgcagcatgaacccagcagctggtgtcccctatgaggaactcagcaacagattgcgacagaacccttgagaacaaaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccaaggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaaaaaaaaaaaaaaaaaaaaaaaaaaaaaaaaaa

SEQ ID NO: 7 - protein, 13 aa, Homo sapiens
AKLPDATPTELAK

SEQ ID NO: 8 - DNA, 90 bp, Homo sapiens
aaactggcagagcgactaaaagcaaaattgcctgatgccacacccacggaactggcaaagctrgttaacaagyrctcagactttgcctcc

SEQ ID NO: 9 - DNA, 90 bp, Homo sapiens
aaactggcagagcgactaaaagcaaaattgcctgaggccacacccacggaactggcaaagctrgttaacaagyrctcagactttgcctcc

SEQ ID NO: 10 - DNA, 90 bp, Homo sapiens
aaactggcagagcgactaaaagcaaaattgcctgatgccacacccaaggaactggcaaagctrgttaacaagyrctcagactttgcctcc

SEQ ID NO: 11 - protein, 30 aa, Homo sapiens
  misc_feature (25)..(25): Xaa can be any naturally occurring amino acid
KLAERLKAKLPDATPTELAKLVNKXSDFAS

SEQ ID NO: 12 - protein, 30 aa, Homo sapiens
  misc_feature (25)..(25): Xaa can be any naturally occurring amino acid
KLAERLKAKLPEATPTELAKLVNKXSDFAS

SEQ ID NO: 13 - protein, 30 aa, Homo sapiens
  misc_feature (25)..(25): Xaa can be any naturally occurring amino acid
KLAERLKAKLPDATPKELAKLVNKXSDFAS

SEQ ID NO: 14 - protein, 458 aa, Homo sapiens
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKRSDFASNCCSINSPPLYCDSEIDAELKNIL

SEQ ID NO: 15 - protein, 474 aa, Homo sapiens; identical to SEQ ID NO: 1

SEQ ID NO: 16 - DNA, 2024 bp, Homo sapiens
atattaagtaaactttagtgaggaacagcagtggaaaataatctatataccttggctcttttgcagtttgacaaagttaatgattaaaatctcctagattttccactacagtatccccagggtgtctatttaccttgattgatattattttatctcttttgggccaaagataacagccccttgcttctgtgtttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaaaaaaaaaaaaaaaaaaaaaaaaaaaaaaaaaa

SEQ ID NO: 17 - protein, 493 aa, Homo sapiens
MLWSWSEERGGAARLSGRKMKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 18 - DNA, 1832 bp, Homo sapiens
actgttttttcaaagagattgcttactccaccaggagaatgatatctacctattctaccactggtgatgatctggactagtccaactggtaggaaatgctgtggtcttggagtgaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaaaaaaaaaaaaaaaaaaaaaaaaaaaaaaaaaa

SEQ ID NO: 19 - protein, 11 aa, Homo sapiens
LPDATPTELAK

SEQ ID NO: 20 - protein, 13 aa, Homo sapiens
AKLPEATPTELAK

SEQ ID NO: 21 - protein, 11 aa, Homo sapiens
LPEATPTELAK

SEQ ID NO: 22 - protein, 9 aa, Homo sapiens
AKLPDATPK

SEQ ID NO: 23 - protein, 7 aa, Homo sapiens
LPDATPK

SEQ ID NO: 24 - protein, 5 aa, Homo sapiens
DATPT

SEQ ID NO: 25 - protein, 5 aa, Homo sapiens
EATPT

SEQ ID NO: 26 - protein, 5 aa, Homo sapiens
DATPK

SEQ ID NO: 27 - protein, 13 aa, Homo sapiens
EDFTSLSLVLYSR

SEQ ID NO: 28 - protein, 11 aa, Homo sapiens
RTHLPEVFLSK